(19) United States
(12) Patent Application Publication
(10) Pub. No.: US 2023/0242540 A1
(43) Pub. Date: Aug. 3, 2023

(54) COMPOUNDS HAVING ANTICANCER ACTIVITY

(71) Applicant: The Trustees of Princeton University, Princeton, NJ (US)

(72) Inventors: Zemer GITAI, Princeton, NJ (US); Hahn KIM, Princeton, NJ (US); James K. MARTIN, Princeton, NJ (US); Joseph P. SHEEHAN, Princeton, NJ (US); Joshua D. RABINOWITZ, Princeton, NJ (US); Xincheng XU, Princeton, NJ (US); Connor CHAIN, Princeton, NJ (US)

(21) Appl. No.: 18/013,302

(22) PCT Filed: Jul. 1, 2021
(86) PCT No.: PCT/US2021/040171

§ 371 Date: Dec. 28, 2022

Related U.S. Application Data
(60) Provisional application No. 63/047,612, filed on Jul. 2, 2020.

(51) Int. Cl. C07D 487/04; C07F 7/08; A61P 35/00

(52) U.S. Cl. CPC C07D 487/04; C07F 7/081; A61P 35/00

(57) ABSTRACT

In one aspect, compounds and associated pharmaceutical compositions are described herein for the treatment of cancer. In some embodiments, for example, a pharmaceutical composition comprises a compound of Formula (I) in an amount sufficient to exhibit anti cancer activity.

### RELATED APPLICATION DATA

The present application claims priority pursuant to Article 8 of the Patent Cooperation Treaty to U.S. Provisional Patent Application Ser. No. 63/047,612 filed Jul. 2, 2020 which is incorporated herein by reference in its entirety.

### STATEMENT OF GOVERNMENT RIGHTS

This invention was made with government support under Grant No. DP1AI124669 awarded by the National Institutes of Health (NIH). The government has certain rights in the invention.

### FIELD

The present invention relates to anticancer compounds and modes of action associated with the compounds.

### BACKGROUND

Cancer is responsible for a significant number of annual deaths in the United States and worldwide. Many strategies for the treatment of cancer currently exist, such as radiation therapy, chemotherapy, and surgery including the complete removal of cancerous tissue as well as cytoreduction and palliation. In a number of cases, cancer treatment strategies comprise adjuvant therapies such as surgery and chemotherapy. In cytoreductive procedures, for example, any abnormal tissue remaining after the surgery can be treated with chemotherapy.

The inherently destructive nature of cancer therapies often results in harmful side-effects such as damage to healthy, non-cancerous tissues. The cytotoxicity of various chemotherapeutic agents, for example, can result in anemia, alopecia (hair loss), nausea and vomiting, damage to nerves leading to burning, numbness, tingling or shooting pain. Chemotherapy can additionally precipitate immunosuppresion and myelosupression thereby increasing a patient's chances for infection and other disease.

Alternative strategies have also been developed, such as hypothermic technologies employing nanoparticle compositions. Nanoparticle therapies have several disadvantages, including the inability to treat deep tumor tissue and the negative immuno-response of nanoparticles collecting in various areas of the lymphatic system. Accordingly, new cancer treatments are needed which can be effective at nanomolar concentrations.

### SUMMARY

In one aspect, compounds and associated pharmaceutical compositions are described herein for the treatment of cancer. In some embodiments, for example, a pharmaceutical composition comprises a compound of Formula (I) and/or salts thereof:

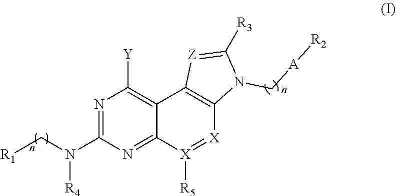

wherein R₁, R₃, R₄ and R₅ are independently selected from the group consisting of hydrogen, alkyl, alkenyl, heteroalkyl, cycloalkyl, heterocycloalkyl, aryl, heteroaryl, imine, cyanoimine, alkylene-aryl, alkylene-heteroaryl, amide, sulfonamide, acid, halo, and urea, wherein the alkyl, alkenyl, heteroalkyl, cycloalkyl, heterocycloalkyl, aryl, heteroaryl, alkylene-aryl, alkylene-heteroaryl, amide and sulfonamide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, cyano, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and

wherein R₂ is selected from the group consisting of alkyl, cycloalkyl, heterocycloalkyl, alkynyl, alkenyl, alkylnylene-alkyl, alkynylene-cycloalkyl, alkynylene-heterocycloalkyl, alkynylene-aryl, alkynylene-heteroaryl, alkynylene-amine, alkynylene-protected amine, alkynylene-alkylsilane, fluoroalkyl, fluoro, bromo, B(OH)₂, nitro, cyano, and alkoxy; and

wherein A is selected from the group consisting of aryl and heteroaryl; and

wherein X and Z are independently selected from the group consisting of C, N, O, S, SO₂, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea and C(O)R₁₂ wherein R₁₂ is selected from the group consisting of hydrogen, alkyl, alkenyl, heteroalkyl, cycloalkyl, heterocycloalkyl, aryl and heteroaryl and wherein R₁₀ and R₁₁ may optionally form a ring structure; and

wherein Y is selected from the group consisting of OH, alkoxy, and NR₁₃R₁₄, wherein R₁₃ and R₁₄ are independently selected from the group consisting of hydrogen, alkyl, heteroalkyl, cycloalkyl, heterocycloalkyl, alkenyl, aryl, heteroaryl, amide, sulfonamide, urea, alkylene-aryl, alkylene-heteroaryl, and C(O)R₁₅ wherein R₁₅ is selected from the group consisting of hydrogen, alkyl, alkenyl, heteroalkyl, cycloalkyl, heterocycloalkyl, aryl and heteroaryl and wherein R₁₃ and R₁₄ may optionally form a ring structure, wherein the aryl, heteroaryl, alkylene-aryl and alkylene heteroaryl are optionally substituted with one or more substituents selected from the group consisting of alkyl, alkenyl, alkynyl, halo, and alkynylene-alkylsilane; and

n is an integer from 0 to 5,

wherein the compound of Formula (I) is present in the pharmaceutical composition in an amount sufficient to exhibit anticancer properties.

In some embodiments, a pharmaceutical composition comprises a compound of Formula (II) and/or salts thereof:

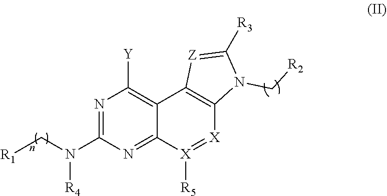

wherein R₁, R₃, R₄ and R₅ are independently selected from the group consisting of hydrogen, alkyl, alkenyl, heteroalkyl, cycloalkyl, heterocycloalkyl, aryl, heteroaryl, alkylene-aryl, alkylene-heteroaryl, amide, sulfonamide, acid, halo, and urea, wherein the alkyl, alkenyl, heteroalkyl, cycloalkyl, heterocycloalkyl, aryl, heteroaryl, alkylene-aryl, alkylene-heteroaryl, amide and sulfonamide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and

wherein R₂ is selected from the group consisting of arylene-alkynyl, heteroarylene-alkynyl, arylene-alkenyl, heteroarylene-alkenyl, alkylnylene-alkyl, alkynylene-cycloalkyl, alkynylene-heterocycloalkyl, alkynylene-aryl, alkynylene-heteroaryl, alkenylene-aryl, alkenylene-heteroaryl, alkynylene-amine, alkynylene-protected amine, and alkynylene-alkylsilane; and

wherein X and Z are independently selected from the group consisting of C, N, O, S, SO₂, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea and C(O)R₁₂ wherein R₁₂ is selected from the group consisting of hydrogen, alkyl, alkenyl, heteroalkyl, cycloalkyl, heterocycloalkyl, aryl and heteroaryl and wherein R₁₀ and R₁₁ may optionally form a ring structure; and

wherein Y is selected from the group consisting of OH and NR₁₂R₁₃, wherein R₁₃ and R₁₄ are independently selected from the group consisting of hydrogen, alkyl, heteroalkyl, cycloalkyl, heterocycloalkyl, alkenyl, aryl, heteroaryl, amide, sulfonamide, urea and C(O)R₁₅ wherein R₁₅ is selected from the group consisting of hydrogen, alkyl, alkenyl, heteroalkyl, cycloalkyl, heterocycloalkyl, aryl and heteroaryl and wherein R₁₃ and R₁₄ may optionally form a ring structure; and n is an integer from 0 to 5,

wherein the compound of Formula (II) is present in the pharmaceutical composition in an amount sufficient to exhibit anticancer properties.

In another aspect, a pharmaceutical composition comprises a compound of Formula (III) and/or salts thereof:

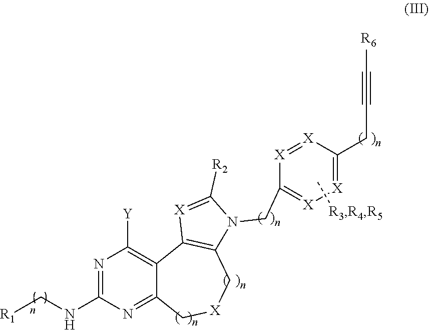

wherein R₁-R₆ are independently selected from the group consisting of hydrogen, alkyl, cycloalkyl, heterocycloalkyl, aryl, heteroaryl, amide, sulfonamide, halo, urea, and —C(O)OR₇, wherein R₇ is selected from the group consisting of hydrogen and alkyl, and wherein each X is independently selected from the group consisting of C, N, O, S, SO₂, and NR₈R₉, wherein R₈ and R₉ are independently selected from the group consisting of hydrogen, alkyl, heteroalkyl, cycloalkyl, heterocycloalkyl, alkenyl, aryl, heteroaryl, amide, sulfonamide, urea and C(O)R₁₀ wherein R₁₀ is selected from the group consisting of hydrogen, alkyl, alkenyl, heteroalkyl, cycloalkyl, heterocycloalkyl, aryl and heteroaryl and wherein R₈ and R₉ may optionally form a ring structure; and wherein Y is selected from the group consisting of OH and NR₁₁R₁₂, wherein R₁₁ and R₁₂ are independently selected from the group consisting of hydrogen, alkyl, heteroalkyl, cycloalkyl, heterocycloalkyl, alkenyl, aryl, heteroaryl, amide, sulfonamide, urea and C(O)R₁₃ wherein R₁₃ is selected from the group consisting of hydrogen, alkyl, alkenyl, heteroalkyl, cycloalkyl, heterocycloalkyl, aryl and heteroaryl and wherein R₁₁ and R₁₂ may optionally form a ring structure; and n is an integer from 0 to 5,

wherein the compound of Formula (III) is present in the pharmaceutical composition in an amount sufficient to exhibit anticancer properties.

In another aspect, a pharmaceutical composition comprises a compound of Formula (IV) and/or salts thereof:

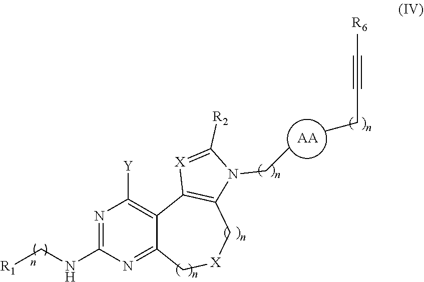

wherein R₁-R₆ are independently selected from the group consisting of hydrogen, alkyl, cycloalkyl, heterocycloalkyl, aryl, heteroaryl, amide, sulfonamide, halo, urea, and —C(O)OR₇, wherein R₇ is selected from the group consisting of hydrogen and alkyl, and wherein each X is independently selected from the group consisting of C, N, O, S, SO₂, and NR₈R₉, wherein R₈ and R₉ are independently selected from the group consisting of hydrogen, alkyl, heteroalkyl, cycloalkyl, heterocycloalkyl, alkenyl, aryl, heteroaryl, amide, sulfonamide, urea and C(O)R₁₀ wherein R₁₀ is selected from the group consisting of hydrogen, alkyl, alkenyl, heteroalkyl, cycloalkyl, heterocycloalkyl, aryl and heteroaryl and wherein R₈ and R₉ may optionally form a ring structure; and wherein Y is selected from the group consisting of OH and NR₁₁R₁₂, wherein R₁₁ and R₁₂ are independently selected from the group consisting of hydrogen, alkyl, heteroalkyl, cycloalkyl, heterocycloalkyl, alkenyl, aryl, heteroaryl, amide, sulfonamide, urea and C(O)R₁₃ wherein R₁₃ is selected from the group consisting of hydrogen, alkyl, alkenyl, heteroalkyl, cycloalkyl, heterocycloalkyl, aryl and heteroaryl and wherein R₁₁ and R₁₂ may optionally form a ring structure; and wherein AA is selected from the group consisting of arylene, heteroarylene, cycloalkylene, and heterocycloalkylene, and n is an integer from 0 to 5,

wherein the compound of Formula (IV) is present in the pharmaceutical composition in an amount sufficient to exhibit anticancer properties.

In another aspect, a pharmaceutical composition comprises a compound of Formula (V) and/or salts thereof:

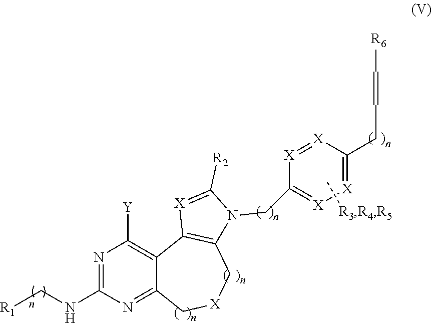

wherein R₁-R₆ are independently selected from the group consisting of hydrogen, alkyl, cycloalkyl, heterocycloalkyl, aryl, heteroaryl, amide, sulfonamide, halo, urea, and —C(O)OR₇, wherein R₇ is selected from the group consisting of hydrogen and alkyl, and wherein each X is independently selected from the group consisting of C, N, O, S, SO₂, and NR₈R₉, wherein R₈ and R₉ are independently selected from the group consisting of hydrogen, alkyl, heteroalkyl, cycloalkyl, heterocycloalkyl, alkenyl, aryl, heteroaryl, amide, sulfonamide, urea and C(O)R₁₀ wherein R₁₀ is selected from the group consisting of hydrogen, alkyl, alkenyl, heteroalkyl, cycloalkyl, heterocycloalkyl, aryl and heteroaryl and wherein R₈ and R₉ may optionally form a ring structure; and wherein Y is selected from the group consisting of OH and NR₁₁R₁₂, wherein R₁₁ and R₁₂ are independently selected from the group consisting of hydrogen, alkyl, heteroalkyl, cycloalkyl, heterocycloalkyl, alkenyl, aryl, heteroaryl, amide, sulfonamide, urea and C(O)R₁₃ wherein R₁₃ is selected from the group consisting of hydrogen, alkyl, alkenyl, heteroalkyl, cycloalkyl, heterocycloalkyl, aryl and heteroaryl and wherein R₁₁ and R₁₂ may optionally form a ring structure; and n is an integer from 0 to 5,

wherein the compound of Formula (V) is present in the pharmaceutical composition in an amount sufficient to exhibit anticancer properties.

In another aspect, a pharmaceutical composition comprises a compound of Formula (VI) and/or salts thereof:

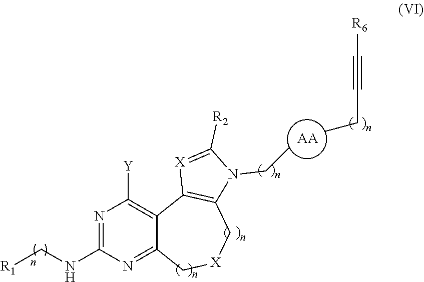

wherein R₁-R₆ are independently selected from the group consisting of hydrogen, alkyl, cycloalkyl, heterocycloalkyl, aryl, heteroaryl, amide, sulfonamide, halo, urea, and —C(O)OR₇, wherein R₇ is selected from the group consisting of hydrogen and alkyl, and wherein each X is independently selected from the group consisting of C, N, O, S, SO₂, and NR₈R₉, wherein R₈ and R₉ are independently selected from the group consisting of hydrogen, alkyl, heteroalkyl, cycloalkyl, heterocycloalkyl, alkenyl, aryl, heteroaryl, amide, sulfonamide, urea and C(O)R₁₀ wherein R₁₀ is selected from the group consisting of hydrogen, alkyl, alkenyl, heteroalkyl, cycloalkyl, heterocycloalkyl, aryl and heteroaryl and wherein R₈ and R₉ may optionally form a ring structure; and wherein Y is selected from the group consisting of OH and NR₁₁R₁₂, wherein R₁₁ and R₁₂ are independently selected from the group consisting of hydrogen, alkyl, heteroalkyl, cycloalkyl, heterocycloalkyl, alkenyl, aryl, heteroaryl, amide, sulfonamide, urea and C(O)R₁₃ wherein R₁₃ is selected from the group consisting of hydrogen, alkyl, alkenyl, heteroalkyl, cycloalkyl, heterocycloalkyl, aryl and heteroaryl and wherein R₁₁ and R₁₂ may optionally form a ring structure; and wherein AA is selected from the group consisting of arylene, heteroarylene, cycloalkylene, and heterocycloalkylene, and n is an integer from 0 to 5,

wherein the compound of Formula (VI) is present in the pharmaceutical composition in an amount sufficient to exhibit anticancer properties.

In some embodiments, one or more compounds falling under one or more of Formulas (I)-(VI) exhibit anticancer properties at nanomolar (nM) concentrations. In some embodiments, for example, the one or more compounds exhibit anticancer properties at a concentration of 0.01 nM to greater than 1 μM.

In another aspect, methods of treating cancerous tissue are described herein. In some embodiments, a method comprises administering to a patient having cancerous tissue one or more compounds of Formula(s) I-VI in therapeutically effective amount. In some embodiments, a therapeutically effective amount stops cancerous cell growth and/or tumor growth. A therapeutically effective amount may also reduce tumor size, in some embodiments.

These and other embodiments are further described in the following detailed description.

### BRIEF DESCRIPTION OF THE DRAWINGS

FIGS. 1A and 1B illustrate inhibition of cancer cell growth by a compound of Formula I described herein according to some embodiments.

FIG. 2 illustrates folate competition from a compound of Formula I according to some embodiments.

FIGS. 3A and 3B illustrate change in tumor volume over time and tumor doubling time, respectively, in response to treatment with a compound of Formula I, according to some embodiments.

FIG. 3C illustrates levels of intermediates in purine synthesis glycineamide ribonucleotide (GAR) and 5-aminoimidazole-4-carboxamide ribonucleotide (AlCAR) in tumors treated with a compound of Formula 1 according to some embodiments.

FIGS. 4A and 4B illustrate change in tumor volume over time and tumor doubling time, respectively, in response to treatment with a compound of Formula I, according to some embodiments.

FIG. 5A illustrate change in tumor volume in response to administration of a compound for Formula I according to some embodiments.

FIG. 5B illustrates pool size of circulating thymidine in response to administration of Compound 1.

FIG. 5C illustrates thymidine ribonucleotide intermediate dUMP and purine intermediates GAR and AlCAR levels in the tumors in response to administration of Compound 1.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents

The term “alkynyl” as used herein, alone or in combination, refers to a straight or branched chain hydrocarbon group having at least one carbon-carbon triple bond and optionally substituted with one or more substituents including, but not limited to, alkyl, cycloalkyl, heterocycloalkyl, aryl, heteroaryl, amine, and/or alkylsilane.

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, oxygen and/or sulfur.

The term “cycloalkyl” as used herein, alone or in combination, refers to a non-aromatic, mono- or multicyclic ring system optionally substituted with one or more ring substituents.

The term “heterocycloalkyl” as used herein, alone or in combination, refers to a non-aromatic, mono- or multicyclic ring system in which one or more of the atoms in the ring system is an element other than carbon, such as nitrogen, oxygen or sulfur, alone or in combination, and wherein the ring system is optionally substituted with one or more ring substituents.

The term “heteroalkyl” as used herein, alone or in combination, refers to an alkyl moiety as defined above, having one or more carbon atoms in the chain, for example one, two or three carbon atoms, replaced with one or more heteroatoms, which may be the same or different, where the point of attachment to the remainder of the molecule is through a carbon atom of the heteroalkyl radical.

The term “alkoxy” as used herein, alone or in combination, refers to the moiety RO—, where R is alkyl or alkenyl defined above.

The term “halo” as used herein, alone or in combination, refers to elements of Group VIIA of the Periodic Table (halogens). Depending on chemical environment, halo can be in a neutral or anionic state. Halo, for example, covers fluoro, chloro, bromo, and iodo.

### I. Pharmaceutical Compositions for Treating Cancer

Various compounds are described herein. As discussed above and further illustrated in the examples below, the compounds can exhibit anticancer properties in some embodiments. The compounds can fall under any one of Formulas I-VI described above. Compounds and/or salt(s) of Formulas I-VI can be individually administered in any amount consistent with precluding or inhibiting growth of cancerous tissue. In some embodiments, one or more of the compounds are administered in an amount or concentration of 0.001 nM to greater than 1 μM. A compounds of any of Formulas I-VI can also be administered in an amount or concentration selected from Table I.

```
TABLE I
Amount of Compound of Formulas I-VI
0.01 nM-500 nM
  0.1 nM-300 nM
  1 nM to 150 nM
0.1 nM to 10 μM
1 nM to 1 μm
```

Additionally, compounds and/or salts of Formulas I-VI can be combined with one another and/or other chemotherapeutic agents or adjuvants in pharmaceutical compositions described herein. Compounds and/or salt(s) of Formulas I-VI. can also be combined with any physiologically suitable carrier or excipient.

The amount or concentration of compounds of Formulas I-VI employed in pharmaceutical compositions described herein can be dependent on the identity and/or nature of the cancer being treated. In some embodiments, compounds of Formulas I-VI are applied against cancer cell lines down-regulating reduced folate carrier (RFC). These cell lines, for example, can be resistant to pemetrexed and/or methotrexate. However, as shown herein, compounds of Formulas I-VI can exhibit higher potency to cell lines down-regulating RFC relative to pemetrexed and/or methotrexate. Compounds of Formulas I-VI, in some embodiments, can inhibit cancer cell growth by disrupting purine and/or thymidine biosynthesis. One or more compounds of Formulas I-VI, for example, may disrupt purine and/or thymidine biosynthesis in pancreatic cancer cells and/or colorectal cancer cells. In some embodiments, compounds of Formulas I-VI can exhibit an IC50 for inhibiting cancer cell growth of 0.001 nM to 1 μM or 0.1 nM to 100 nM. The IC50, in some embodiments, is greater than 1 μM.

### II. Methods of Treating Cancer

In another aspect, methods of treating cancerous tissue are described herein. In some embodiments, a method comprises administering to a patient having cancerous tissue one or more compounds of Formula(s) I-VI or salts thereof in therapeutically effective amount. In some embodiments, a therapeutically effective amount stops or inhibits cancerous cell growth and/or tumor growth. A therapeutically effective amount may also reduce tumor size, in some embodiments. In some embodiments, a compound of any of Formulas I-VI or salt thereof is administered in an amount selected from Table I. In some embodiments, a combination of two or more compounds of any of Formulas I-VI can be employed in treating cancerous cells and tissue. Moreover, compounds of Formulas I-VI can be combined with any adjuvants or other chemotherapeutic agents for the treatment of cancer.

These and other embodiments are further illustrated in the following non-limiting examples.

### EXAMPLES—COMPOUNDS EXHIBITING ANTICANCER ACTIVITY

One or more of the following compounds according to at least one of Formulas I-VI, in some embodiments, exhibit anticancer activity. The compounds were prepared according to the following general reaction scheme. Common solvents were purified before use. All reagents were reagent grade and purified where necessary. Reactions were monitored by thin-layer chromatography (TLC) using Whatman precoated silica gel plates. Flash column chromatography was performed over ultrapure silica gel (200-400 mesh) from Merck. ¹H NMR spectra were recorded on a Bruker AVANCE 300 (300 MHz), 400 MHz or 500 MHz spectrometer. Multiplicities for ¹H NMR are designated as s=singlet, d=doublet, t=triplet, q=quartet, quint=quintet, sext=sextet, dd=doublet of doublets, dt=doublet of triplets, m=multiplet, and br=broad. Electrospray impact (ESI) mass spectra were recorded on ISQEC mass spectrometer.

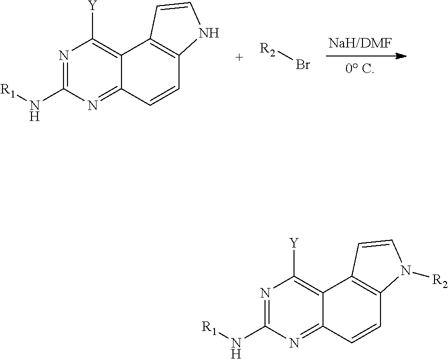

To a stirred solution of 7H-Pyrrolo[3, 2-f]quinazoline-1,3-diamine (1.0 mmol) in dry DMF (20 mL) was added NaH (1.2 mmol). The resulting reaction mixture was stirred at 0° C. for 0.5 h. Then corresponding bromide (1.5 mmol) was added. The reaction mixture was stirred at 0° C. for 1 h. The solvent was removed under reduced pressure and the residue was purified by column chromatography on silica gel, eluting with 10:1 DCM:MeOH containing 1% Et₃N to give the desired compound as a solid. The following specific compounds were synthesized according to the foregoing procedure.

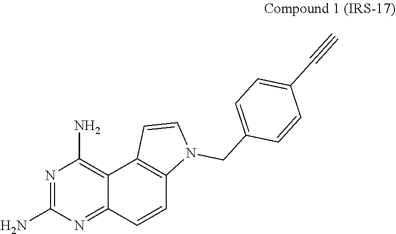 [SMILES: C#Cc1ccc(Cn2ccc3c4c(N)nc(N)nc4ccc32)cc1]

¹H NMR (500 MHz, DMSO-d₆) δ 7.71 (d, J=9.0 Hz, 1H), 7.62 (d, J=3.0 Hz, 1H), 7.41 (d, J=8.0 Hz, 2H), 7.15 (d, J=8.0 Hz, 2H), 7.12 (d, J=3.0 Hz, 1H), 7.03 (d, J=9.0 Hz, 1H), 6.78 (s, 2H), 5.89 (s, 2H), 5.53 (s, 2H), 4.14 (s, 1H). MS (ESI): [M+H⁺] 314.12.

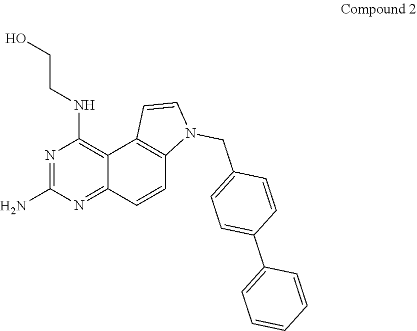 [SMILES: Nc1nc(NCCO)c2c(ccc3c2ccn3Cc2ccc(-c3ccccc3)cc2)n1]

¹H NMR (400 MHz, DMSO-d₆) δ 7.92 (d, J=9.0 Hz, 1H), 7.85 (d, J=3.2 Hz, 1H), 7.65-7.56 (m, 5H), 7.47-7.40 (m, 2H), 7.37-7.31 (m, 1H), 7.29-7.24 (m, 2H), 7.22-7.11 (m, 2H), 6.74 (s, 2H), 5.60 (s, 2H), 3.84-3.66 (m, 4H).

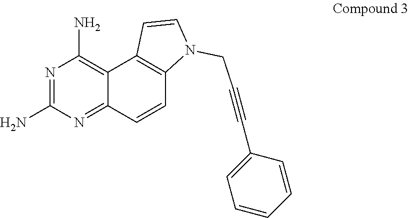 [SMILES: Nc1nc(N)c2c(ccc3c2ccn3CC#Cc2ccccc2)n1]

¹H NMR (300 MHz, DMSO-d₆) δ 7.88 (d, J=9.0 Hz, 1H), 7.58 (d, J=3.3 Hz, 1H), 7.46-7.33 (m, 5H), 7.17-7.06 (m, 2H), 6.70 (s, 2H), 5.72 (s, 2H), 5.43 (s, 2H).

MS(ESI): [M+H+]314.12.

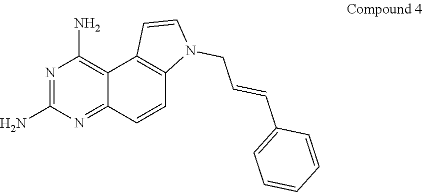 [SMILES: Nc1nc(N)c2c(ccc3c2ccn3C/C=C/c2ccccc2)n1]

¹H NMR (300 MHz, DMSO-d₆) δ 7.80 (d, J=9.0 Hz, 1H), 7.52 (d, J=3.0 Hz, 1H), 7.43-7.37 (m, 2H), 7.34-7.26 (m, 2H), 7.26-7.22 (m, 1H), 7.08 (d, J=3.0 Hz, 1H), 7.05 (d, J=9.0 Hz, 1H), 6.67 (s, 2H), 6.52-6.46 (m, 2H), 5.67 (s, 2H), 5.09-5.01 (m, 2H).

MS(ESI): [M+H+]316.15.

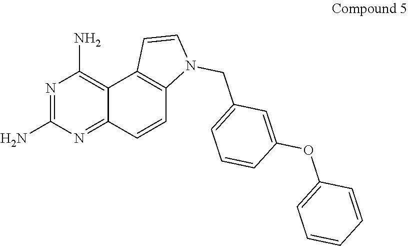 [SMILES: Nc1nc(N)c2c(ccc3c2ccn3Cc2cccc(Oc3ccccc3)c2)n1]

¹H NMR (300 MHz, DMSO-d₆) δ 7.71 (d, J=9.0 Hz, 1H), 7.60 (d, J=3.0 Hz, 1H), 7.39-7.25 (m, 3H), 7.13 (d, J=7.2 Hz, 1H), 7.08 (d, J=3.0 Hz, 1H), 7.02 (d, J=9.0 Hz, 1H), 6.97-6.89 (m, 3H), 6.87-6.80 (m, 2H), 6.68 (s, 2H), 5.69 (s, 2H), 5.50 (s, 2H).

MS(ESI): [M+H+] 382.12.

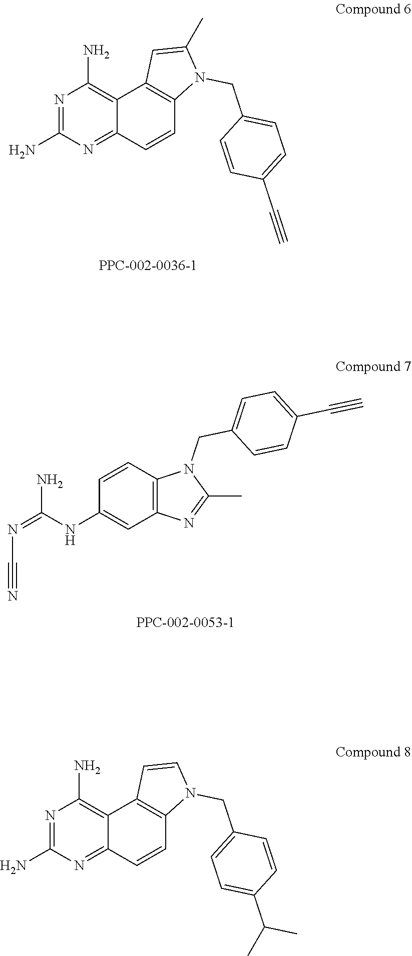 [SMILES: C#Cc1ccc(Cn2c(C)cc3c4c(N)nc(N)nc4ccc32)cc1 ; C#Cc1ccc(Cn2c(C)nc3cc(N/C(N)=N\C#N)ccc32)cc1 ; CC(C)c1ccc(Cn2ccc3c4c(N)nc(N)nc4ccc32)cc1]

¹H NMR (500 MHz, DMSO-d₆) δ 7.73 (d, J=9.0 Hz, 1H), 7.59 (d, J=3.0 Hz, 1H), 7.15 (d, J=8.0 Hz, 2H), 7.11 (d, J=8.0 Hz, 2H), 7.06 (d, J=3.0 Hz, 1H), 7.01 (d, J=8.9 Hz, 1H), 6.65 (s, 2H), 5.65 (s, 2H), 5.43 (s, 2H), 2.80 (p, J=7.0 Hz, 1H), 1.12 (d, J=6.9 Hz, 6H).

MS(ESI): [M+H+]332.42.

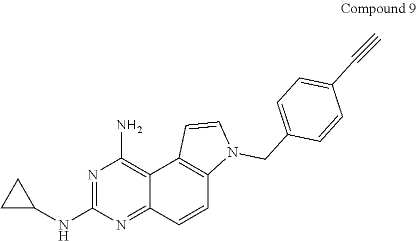 [SMILES: C#Cc1ccc(Cn2ccc3c4c(N)nc(NC5CC5)nc4ccc32)cc1]

¹H NMR (300 MHz, DMSO-d₆) δ 7.70 (d, J=9.0 Hz, 1H), 7.62 (d, J=3.0 Hz, 1H), 7.41 (d, J=8.2 Hz, 2H), 7.14 (d, J=8.2 Hz, 2H), 7.11 (d, J=3.0 Hz, 1H), 7.09 (d, J=9.0 Hz, 1H), 6.68 (s, 2H), 6.47-6.30 (m, 1H), 5.53 (s, 2H), 4.16 (s, 1H), 2.84-2.70 (m, 1H), 0.68-0.57 (m, 2H), 0.48-0.39 (m, 2H).

MS(ESI): [M+H+] 354.15.

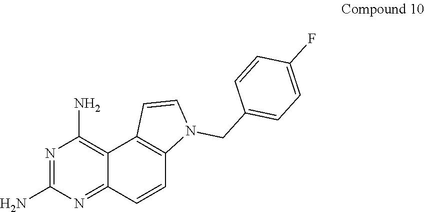 [SMILES: Nc1nc(N)c2c(ccc3c2ccn3Cc2ccc(F)cc2)n1]

¹H NMR (500 MHz, DMSO-d₆) δ 8.08 (d, J=9.0 Hz, 1H), 7.91 (d, J=3.0 Hz, 1H), 7.60 (s, 2H), 7.37 (d, J=3.0 Hz, 1H), 7.31-7.24 (m, 2H), 7.21 (d, J=9.0 Hz, 1H), 7.18-7.11 (m, 2H), 5.56 (s, 2H).

MS(ESI): [M+H+] 308.15.

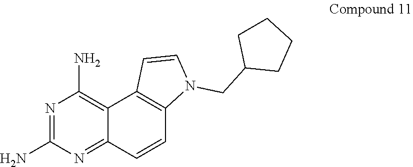 [SMILES: Nc1nc(N)c2c(ccc3c2ccn3CC2CCCC2)n1]

¹H NMR (500 MHz, DMSO-d₆) δ 7.78 (d, J=9.0 Hz, 1H), 7.47 (d, J=3.0 Hz, 1H), 7.05 (d, J=9.0 Hz, 1H), 7.00 (d, J=3.0 Hz, 1H), 6.68 (s, 2H), 5.69 (s, 2H), 4.13 (d, J=7.5 Hz, 2H), 2.42-2.29 (m, 1H), 1.66-1.58 (m, 2H), 1.58-1.52 (m, 2H), 1.51-1.44 (m, 2H), 1.28-1.20 (m, 2H).

MS(ESI): [M+H+] 282.24.

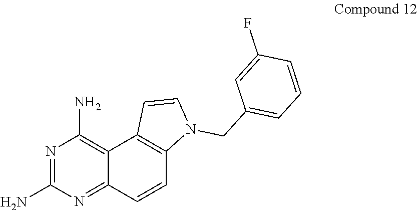 [SMILES: Nc1nc(N)c2c(ccc3c2ccn3Cc2cccc(F)c2)n1]

¹H NMR (400 MHz, DMSO-d₆) δ 7.92-7.82 (m, 1H), 7.80-7.72 (m, 1H), 7.39-7.31 (m, 1H), 7.25-7.18 (m, 1H), 7.14-7.05 (m, 2H), 7.04-6.96 (m, 2H), 5.55 (s, 2H).

MS(ESI): [M+H+] 308.06.

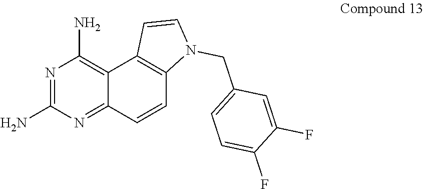 [SMILES: Nc1nc(N)c2c(ccc3c2ccn3Cc2ccc(F)c(F)c2)n1]

¹H NMR (400 MHz, DMSO-d₆) δ 7.75 (d, J=9.0 Hz, 1H), 7.63 (d, J=3.0 Hz, 1H), 7.41-7.34 (m, 1H), 7.34-7.26 (m, 1H), 7.11 (d, J=3.0 Hz, 1H), 7.03 (d, J=9.0 Hz, 1H), 7.02-6.99 (m, 1H), 6.72 (s, 2H), 5.73 (s, 2H), 5.49 (s, 2H).

MS(ESI): [M+H+] 326.10.

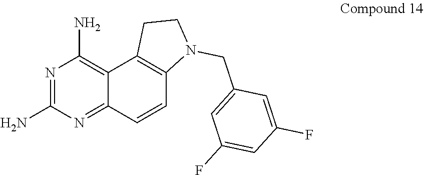 [SMILES: Nc1nc(N)c2c3c(ccc2n1)N(Cc1cc(F)cc(F)c1)CC3]

¹H NMR (400 MHz, DMSO-d₆) δ 7.76 (d, J=9.0 Hz, 1H), 7.66 (d, J=3.2 Hz, 1H), 7.18-7.09 (m, 2H), 7.05 (d, J=9.0 Hz, 1H), 6.90-6.84 (m, 2H), 6.78 (s, 2H), 5.81 (s, 2H), 5.54 (s, 2H).

MS(ESI): [M+H+] 326.10.

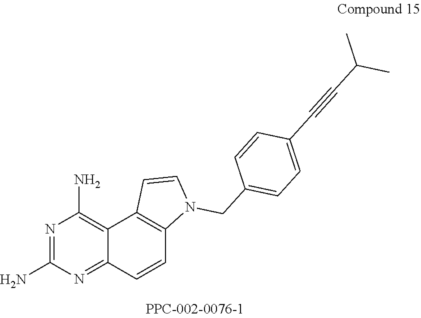 [SMILES: CC(C)C#Cc1ccc(Cn2ccc3c4c(N)nc(N)nc4ccc32)cc1]

¹H NMR (400 MHz, DMSO-d₆) δ 7.69 (d, J=9.0 Hz, 1H), 7.60 (d, J=3.2 Hz, 1H), 7.27 (d, J=8.0 Hz, 2H), 7.09 (d, J=8.0 Hz, 3H), 7.01 (d, J=9.0 Hz, 1H), 6.77 (s, 2H), 5.78 (s, 2H), 5.48 (s, 2H), 2.81-2.65 (m, 1H), 1.15 (d, J=6.8 Hz, 6H).

MS (ESI): [M+H⁺] 356.20

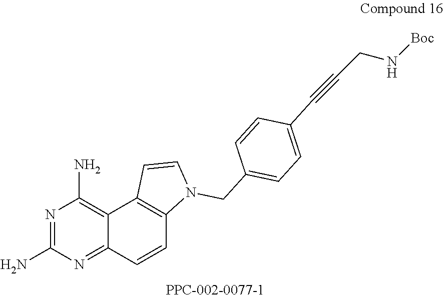 [SMILES: CC(C)(C)OC(=O)NCC#Cc1ccc(Cn2ccc3c4c(N)nc(N)nc4ccc32)cc1]

¹H NMR (500 MHz, DMSO-d₆) δ 7.70 (d, J=9.0 Hz, 1H), 7.61 (d, J=3.1 Hz, 1H), 7.34-7.29 (m, 2H), 7.29 (s, 1H), 7.18-7.05 (m, 3H), 7.01 (d, J=9.0 Hz, 1H), 6.79 (s, 2H), 5.79 (s, 2H), 5.49 (s, 2H), 3.91 (d, J=6.0 Hz, 2H), 1.35 (s, 9H).

MS (ESI): [M+H⁺] 443.53

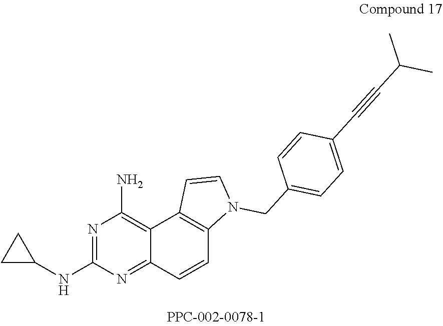 [SMILES: CC(C)C#Cc1ccc(Cn2ccc3c4c(N)nc(NC5CC5)nc4ccc32)cc1]

¹H NMR (400 MHz, DMSO-d₆) δ 7.68 (m, 1H), 7.64-7.59 (m, 1H), 7.34-7.26 (m, 2H), 7.15-7.06 (m, 4H), 6.67 (s, 2H), 6.37 (s, 1H), 5.51 (s, 2H), 2.83-2.74 (m, 1H), 1.18 (d, J=6.9 Hz, 6H), 0.63 (m, 2H), 0.51-0.37 (m, 2H).

MS (ESI): [M+H⁺] 396.50

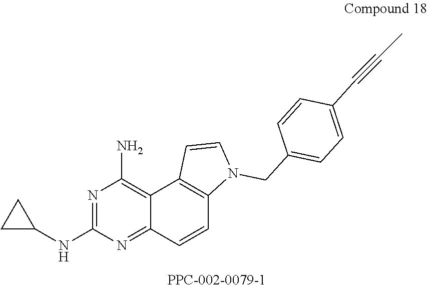 [SMILES: CC#Cc1ccc(Cn2ccc3c4c(N)nc(NC5CC5)nc4ccc32)cc1]

¹H NMR (400 MHz, DMSO-d₆) δ 7.73 (d, J=9.2 Hz, 1H), 7.69-7.60 (m, 1H), 7.32 (d, J=6.4 Hz, 2H), 7.15-7.07 (m, 4H), 5.51 (s, 2H), 2.88-2.72 (m, 1H), 0.86 (t, J=6.4 Hz, 2H), 0.64 (dd, J=6.4, 2.4 Hz, 2H).

MS (ESI): [M+H⁺] 368.45

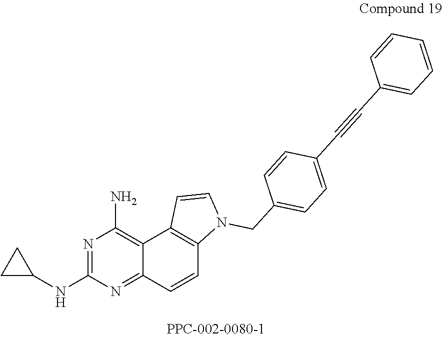 [SMILES: Nc1nc(NC2CC2)nc2ccc3c(ccn3Cc3ccc(C#Cc4ccccc4)cc3)c12]

¹H NMR (400 MHz, DMSO-d₆) δ 7.74 (d, J=8.8 Hz, 1H), 7.66 (d, J=3.2 Hz, 1H), 7.55-7.49 (m, 4H), 7.42 (d, J=3.2 Hz, 3H), 7.20 (d, J=8.0 Hz, 2H), 7.15-7.11 (m, 2H), 6.75 (s, 2H), 6.46 (s, 1H), 5.57 (s, 2H), 2.79 (m, 1H), 0.64 (m, 2H), 0.52-0.40 (m, 2H).

MS (ESI): [M+H⁺] 430.49

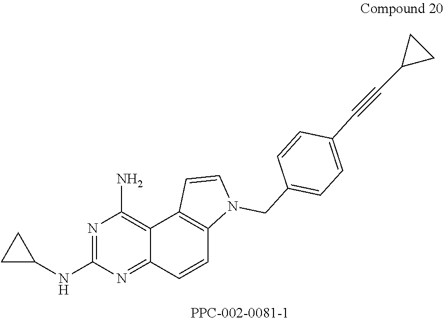 [SMILES: Nc1nc(NC2CC2)nc2ccc3c(ccn3Cc3ccc(C#CC4CC4)cc3)c12]

¹H NMR (400 MHz, DMSO-d₆) δ 7.71 (d, J=9.2 Hz, 1H), 7.61 (d, J=3.2 Hz, 1H), 7.33-7.26 (m, 2H), 7.16-7.06 (m, 4H), 6.69 (s, 2H), 6.40 (s, 1H), 5.50 (s, 2H), 2.79 (m, 1H), 1.51 (m, 1H), 0.91-0.81 (m, 4H), 0.73-0.59 (m, 4H)

MS (ESI): [M+H⁺] 394.19

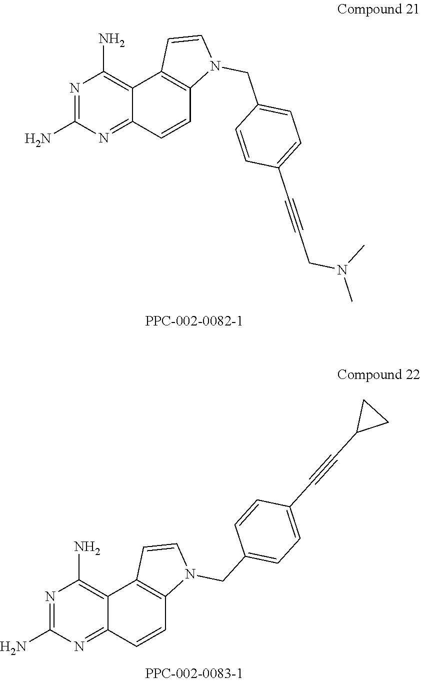 [SMILES: CN(C)CC#Cc1ccc(Cn2ccc3c4c(N)nc(N)nc4ccc32)cc1 ; Nc1nc(N)c2c(ccc3c2ccn3Cc2ccc(C#CC3CC3)cc2)n1]

¹H NMR (400 MHz, DMSO-d₆) δ 7.70 (d, J=8.8 Hz, 1H), 7.61 (d, J=3.2 Hz, 1H), 7.29 (d, J=8.0 Hz, 2H), 7.17-7.08 (m, 3H), 7.02 (d, J=8.8 Hz, 1H), 6.73 (s, 2H), 5.76 (s, 2H), 5.50 (s, 2H), 1.50 (m, 1H), 0.91-0.79 (m, 2H), 0.72-0.64 (m, 2H).

MS (ESI): [M+H⁺] 354.46

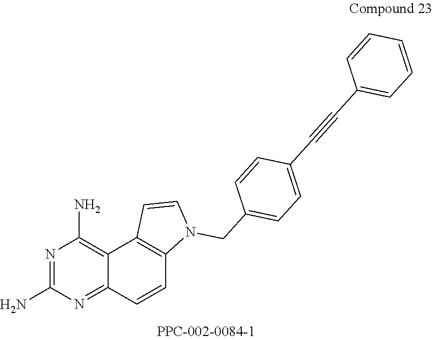 [SMILES: Nc1nc(N)c2c(ccc3c2ccn3Cc2ccc(C#Cc3ccccc3)cc2)n1]

¹H NMR (400 MHz, DMSO-d₆) δ 7.80 (d, J=8.8 Hz, 1H), 7.71 (d, J=3.2 Hz, 1H), 7.57-7.46 (m, 4H), 7.42 (q, J=2.8 Hz, 3H), 7.25-7.16 (m, 3H), 7.08 (d, J=8.8 Hz, 1H), 6.13 (s, 2H), 5.58 (s, 2H).

MS (ESI): [M+H⁺] 390.41

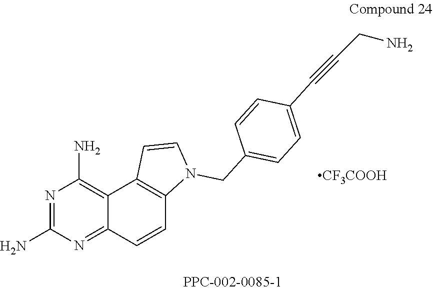

¹H NMR (400 MHz, DMSO-d₆) δ 8.06 (d, J=9.0 Hz, 1H), 7.93 (d, J=3.2 Hz, 1H), 7.67 (s, 2H), 7.47-7.37 (m, 3H), 7.22 (dd, J=9.0, 2.4 Hz, 3H), 5.64 (s, 2H), 3.98 (s, 2H).

MS (ESI): [M+H⁺] 343.38

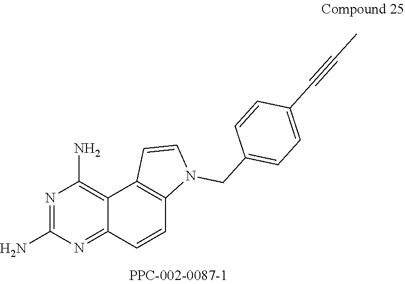 [SMILES: CC#Cc1ccc(Cn2ccc3c4c(N)nc(N)nc4ccc32)cc1]

¹H NMR (400 MHz, DMSO-d₆) δ 7.75 (d, J=9.0 Hz, 1H), 7.65 (d, J=3.2 Hz, 1H), 7.37-7.26 (m, 2H), 7.17-7.10 (m, 3H), 7.04 (d, J=9.0 Hz, 1H), 6.88 (s, 2H), 5.93 (s, 2H), 5.51 (s, 2H), 2.00 (s, 3H).

MS (ESI): [M+H⁺] 328.37

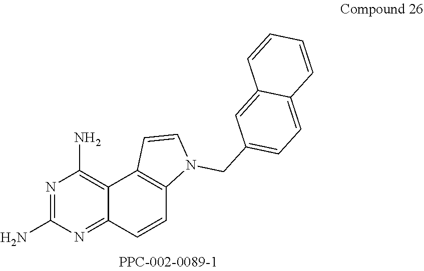 [SMILES: Nc1nc(N)c2c(ccc3c2ccn3Cc2ccc3ccccc3c2)n1]

¹H NMR (300 MHz, DMSO-d₆) δ 7.90-7.79 (m, 4H), 7.77 (d, J=3.2 Hz, 1H), 7.72 (s, 1H), 7.52-7.43 (m, 2H), 7.33 (dd, J=8.5, 1.8 Hz, 1H), 7.19 (d, J=3.2 Hz, 1H), 7.06 (d, J=9.0 Hz, 1H), 6.22 (s, 2H), 5.68 (s, 2H).

MS (ESI): [M+H⁺] 340.35

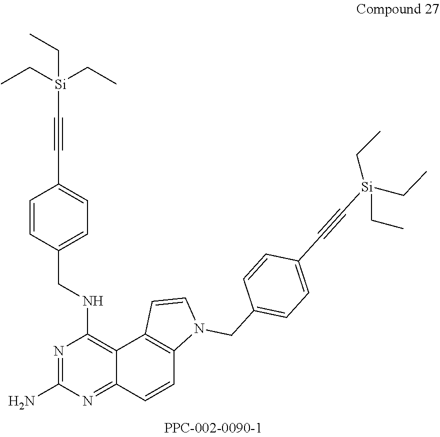 [SMILES: CC[Si](C#Cc1ccc(CNc2nc(N)nc3ccc4c(ccn4Cc4ccc(C#C[Si](CC)(CC)CC)cc4)c23)cc1)(CC)CC]

¹H NMR (500 MHz, DMSO-d₆) δ 7.70 (s, 1H), 7.65 (d, J=3.5 Hz, 1H), 7.41-7.34 (m, 6H), 7.31 (s, 1H), 7.23 (d, J=3.0 Hz, 1H), 7.14 (d, J=8.0 Hz, 2H), 5.86 (s, 2H), 5.51 (s, 2H), 4.83 (d, J=6.0 Hz, 2H), 0.95 (m, 18H), 0.59 (m, 12H).

MS (ESI): [M+H⁺] 657.03

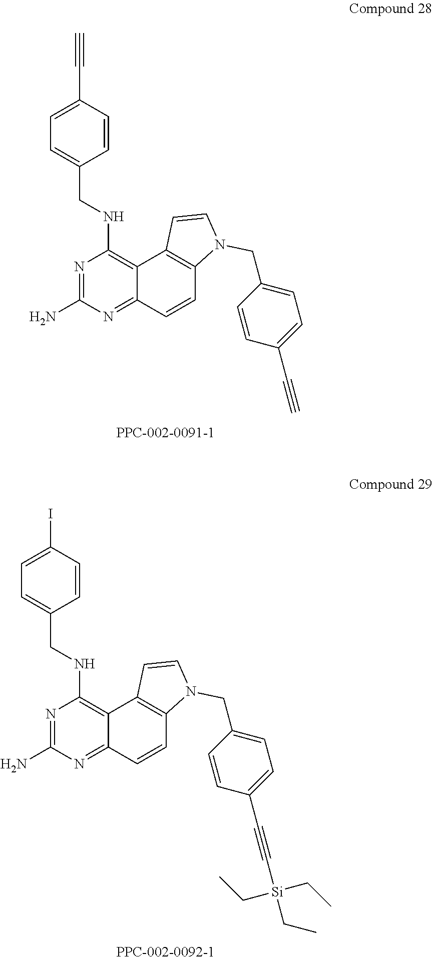 [SMILES: C#Cc1ccc(CNc2nc(N)nc3ccc4c(ccn4Cc4ccc(C#C)cc4)c23)cc1 ; CC[Si](C#Cc1ccc(Cn2ccc3c4c(NCc5ccc(I)cc5)nc(N)nc4ccc32)cc1)(CC)CC]

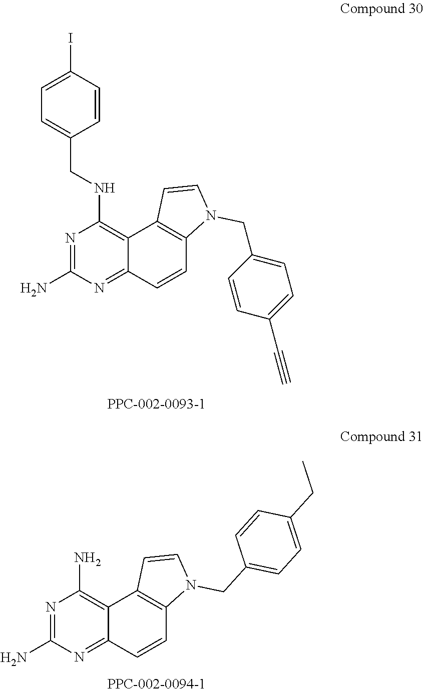 [SMILES: C#Cc1ccc(Cn2ccc3c4c(NCc5ccc(I)cc5)nc(N)nc4ccc32)cc1 ; CCc1ccc(Cn2ccc3c4c(N)nc(N)nc4ccc32)cc1]

¹H NMR (400 MHz, DMSO-d₆) δ 7.79 (d, J=9.0 Hz, 1H), 7.65 (d, J=3.2 Hz, 1H), 7.13 (t, J=3.2 Hz, 5H), 7.05 (d, J=9.0 Hz, 1H), 6.92 (s, 2H), 5.98 (s, 2H), 5.46 (s, 2H), 2.55 (m, 2H), 1.12 (t, J=7.6 Hz, 3H).

MS (ESI): [M+H⁺] 318.36

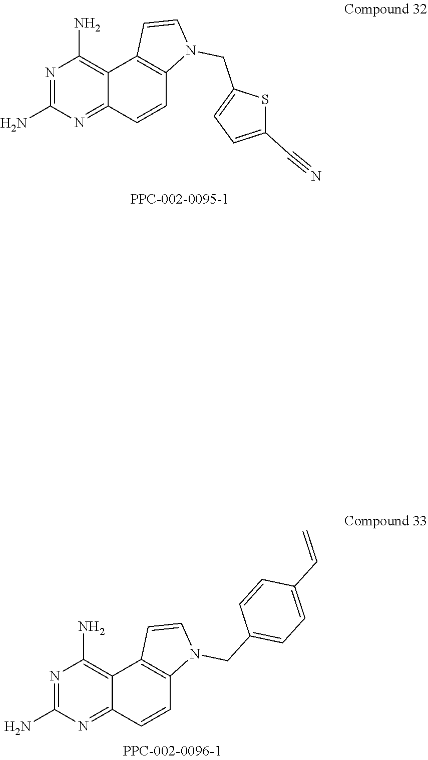 [SMILES: N#Cc1ccc(Cn2ccc3c4c(N)nc(N)nc4ccc32)s1 ; C=Cc1ccc(Cn2ccc3c4c(N)nc(N)nc4ccc32)cc1]

¹H NMR (500 MHz, DMSO-d₆) δ 7.71 (d, J=9.0 Hz, 1H), 7.60 (d, J=3.0 Hz, 1H), 7.41-7.34 (m, 2H), 7.13 (d, J=8.2 Hz, 2H), 7.07 (d, J=3.0 Hz, 1H), 7.00 (d, J=9.0 Hz, 1H), 6.74 (s, 2H), 6.65 (dd, J=17.6, 11.0 Hz, 1H), 5.81-5.68 (m, 3H), 5.46 (s, 2H), 5.19 (dd, J=10.9, 1.0 Hz, 1H).

MS (ESI): [M+H⁺] 316.40

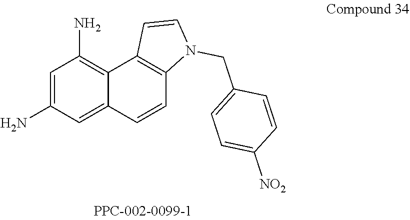 [SMILES: Nc1cc(N)c2c(ccc3c2ccn3Cc2ccc([N+](=O)[O-])cc2)c1]

¹H NMR (400 MHz, DMSO-d₆) δ 8.22-8.17 (m, 2H), 7.96 (d, J=9.0 Hz, 1H), 7.91 (d, J=3.2 Hz, 1H), 7.43-7.35 (m, 3H), 7.19 (d, J=9.0 Hz, 1H), 7.12 (s, 2H), 5.76 (s, 2H).

MS (ESI): [M+H⁺] 335.08

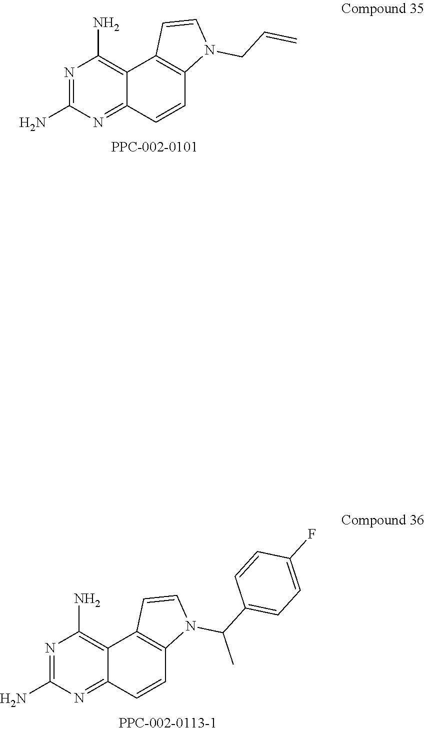 [SMILES: C=CCn1ccc2c3c(N)nc(N)nc3ccc21 ; CC(c1ccc(F)cc1)n1ccc2c3c(N)nc(N)nc3ccc21]

¹H NMR (400 MHz, DMSO-d₆) δ 7.80 (d, J=3.2 Hz, 1H), 7.74 (d, J=8.8 Hz, 1H), 7.33-7.23 (m, 2H), 7.19-7.07 (m, 3H), 7.01 (d, J=8.8 Hz, 1H), 6.80 (s, 2H), 5.95 (q, J=6.8 Hz, 1H), 5.81 (s, 2H), 1.90 (d, J=6.8 Hz, 3H).

MS (ESI): [M+H⁺] 322.32

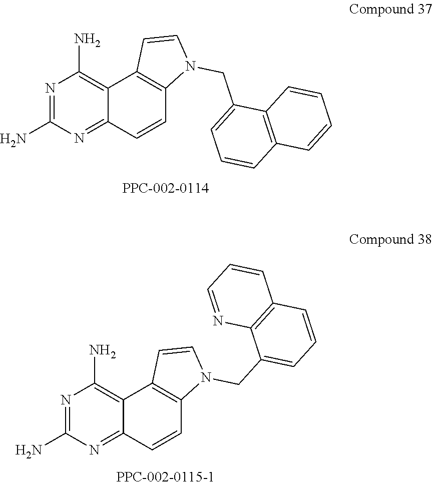 [SMILES: Nc1nc(N)c2c(ccc3c2ccn3Cc2cccc3ccccc23)n1 ; Nc1nc(N)c2c(ccc3c2ccn3Cc2cccc3cccnc23)n1]

¹H NMR (400 MHz, DMSO-d₆) δ 9.07 (dd, J=4.0, 2.0 Hz, 1H), 8.43 (dd, J=8.4, 2.0 Hz, 1H), 7.92 (d, J=8.0 Hz, 1H), 7.80 (d, J=8.8 Hz, 1H), 7.73 (d, J=3.2 Hz, 1H), 7.65 (dd, J=8.4, 4.4 Hz, 1H), 7.47 (t, J=8.0 Hz, 1H), 7.16 (d, J=3.2 Hz, 1H), 7.03 (dd, J=8.4, 4.4 Hz, 2H), 6.92 (s, 2H), 6.15 (s, 2H), 5.95 (s, 2H).

MS (ESI): [M+H⁺] 341.33

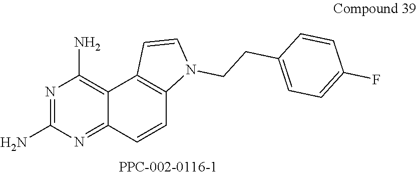 [SMILES: Nc1nc(N)c2c(ccc3c2ccn3CCc2ccc(F)cc2)n1]

¹H NMR (400 MHz, DMSO-d₆) δ 7.78 (d, J=8.8 Hz, 1H), 7.37 (d, J=3.2 Hz, 1H), 7.25-7.14 (m, 2H), 7.12-6.94 (m, 4H), 6.73 (s, 2H), 5.79 (s, 2H), 4.47 (t, J=7.2 Hz, 2H), 3.08 (t, J=7.2 Hz, 2H).

MS (ESI): [M+H⁺] 341.33

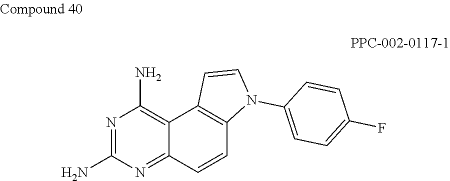 [SMILES: Nc1nc(N)c2c(ccc3c2ccn3-c2ccc(F)cc2)n1]

¹H NMR (300 MHz, Methanol-d₄) δ 7.78 (dd, J=9.1, 0.9 Hz, 1H), 7.66 (d, J=3.3 Hz, 1H), 7.62-7.55 (m, 2H), 7.41-7.30 (m, 2H), 7.25-7.18 (m, 2H).

MS (ESI): [M+H⁺] 294.30

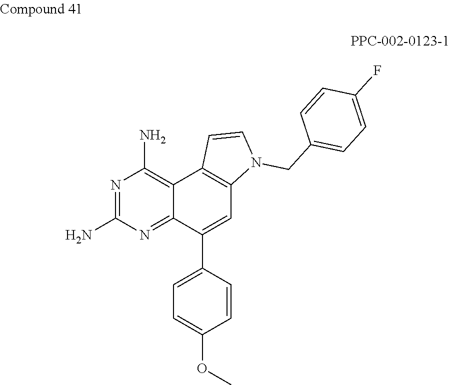 [SMILES: COc1ccc(-c2cc3c(ccn3Cc3ccc(F)cc3)c3c(N)nc(N)nc23)cc1]

¹H NMR (400 MHz, DMSO-d₆) δ 7.74 (s, 1H), 7.65 (d, J=3.2 Hz, 1H), 7.55-7.46 (m, 2H), 7.30-7.20 (m, 2H), 7.20-7.09 (m, 3H), 7.01-6.94 (m, 2H), 6.83 (s, 2H), 5.71 (s, 2H), 5.54 (s, 2H), 3.80 (s, 3H).

MS (ESI): [M+H⁺] 414.48

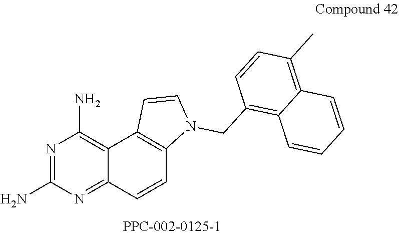 [SMILES: Cc1ccc(Cn2ccc3c4c(N)nc(N)nc4ccc32)c2ccccc12]

¹H NMR (400 MHz, DMSO-d₆) δ 8.22-8.13 (m, 1H), 8.12-8.03 (m, 1H), 7.72 (d, J=9.2 Hz, 1H), 7.66-7.57 (m, 2H), 7.48 (d, J=3.2 Hz, 1H), 7.24 (d, J=7.2 Hz, 1H), 7.11 (d, J=3.2 Hz, 1H), 7.02 (d, J=9.2 Hz, 1H), 6.70 (s, 2H), 6.68 (s, 1H), 5.97 (s, 2H), 5.70 (s, 2H), 2.62 (s, 3H).

MS (ESI): [M+H⁺] 354.44

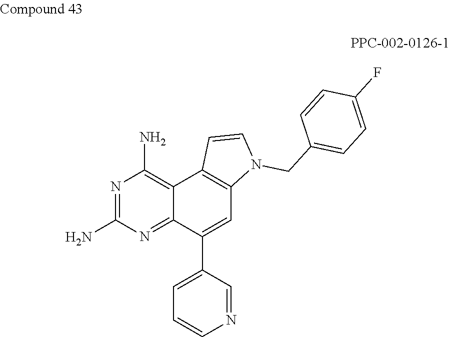 [SMILES: Nc1nc(N)c2c(n1)c(-c1cccnc1)cc1c2ccn1Cc1ccc(F)cc1]

¹H NMR (400 MHz, Methanol-d₄) δ 8.70 (dd, J=2.2, 0.8 Hz, 1H), 8.57 (dd, J=4.8, 1.6 Hz, 1H), 8.04 (dt, J=8.0, 2.0 Hz, 1H), 7.83 (d, J=0.8 Hz, 1H), 7.66 (d, J=3.2 Hz, 1H), 7.56 (m, 1H), 7.29-7.18 (m, 2H), 7.13 (dd, J=3.2, 0.8 Hz, 1H), 7.10-7.00 (m, 2H), 5.56 (s, 2H).

MS (ESI): [M+H⁺] 385.41

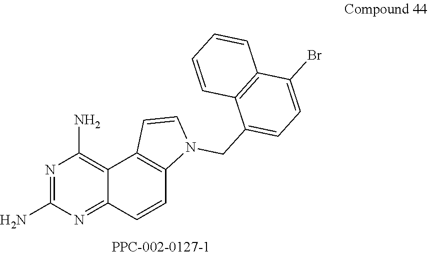 [SMILES: Nc1nc(N)c2c(ccc3c2ccn3Cc2ccc(Br)c3ccccc23)n1]

¹H NMR (400 MHz, DMSO-d₆) δ 8.25 (m, 2H), 7.79-7.70 (m, 4H), 7.55 (d, J=3.1 Hz, 1H), 7.17 (d, J=3.1 Hz, 1H), 7.03 (d, J=8.9 Hz, 1H), 6.76 (s, 2H), 6.54 (d, J=7.7 Hz, 1H), 6.04 (s, 2H), 5.76 (s, 2H).

MS (ESI): [M+H⁺] 418.31

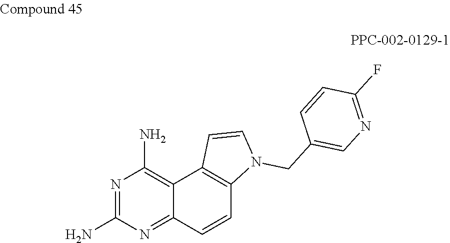 [SMILES: Nc1nc(N)c2c(ccc3c2ccn3Cc2ccc(F)nc2)n1]

¹H NMR (400 MHz, DMSO-d₆) δ 8.24 (d, J=2.4 Hz, 1H), 7.99 (d, J=8.9 Hz, 1H), 7.82 (t, J=5.5 Hz, 2H), 7.47 (s, 2H), 7.25 (d, J=3.2 Hz, 1H), 7.15 (dd, J=8.9, 2.4 Hz, 2H), 6.58 (s, 2H), 5.59 (s, 2H).

MS (ESI): [M+H⁺] 309.36

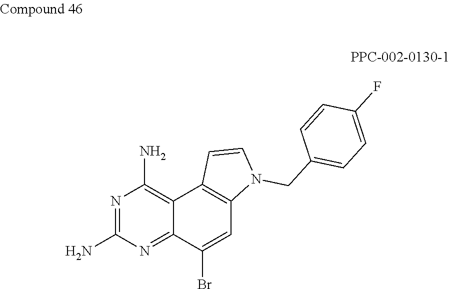 [SMILES: Nc1nc(N)c2c(n1)c(Br)cc1c2ccn1Cc1ccc(F)cc1]

¹H NMR (400 MHz, DMSO-d₆) δ 8.21 (s, 1H), 7.66 (d, J=3.2 Hz, 1H), 7.29-7.20 (m, 2H), 7.20-7.09 (m, 3H), 6.85 (s, 2H), 5.94 (s, 2H), 5.51 (s, 2H).

MS (ESI): [M+H⁺] 386.21

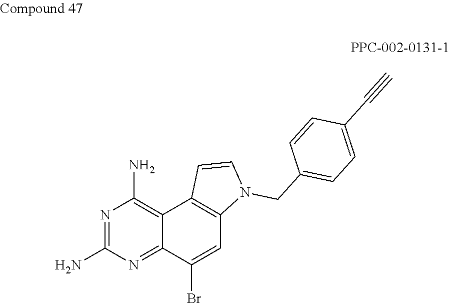 [SMILES: C#Cc1ccc(Cn2ccc3c4c(N)nc(N)nc4c(Br)cc32)cc1]

¹H NMR (400 MHz, Chloroform-d+MeOD) δ 7.82 (d, J=1.2 Hz, 1H), 7.30 (d, J=8.0 Hz, 2H), 7.25-7.23 (m, 1H), 6.90 (d, J=8.0 Hz, 2H), 6.72 (d, J=3.2 Hz, 1H), 5.29 (s, 2H), 3.03 (s, 1H).

MS (ESI): [M+H⁺] 393.25

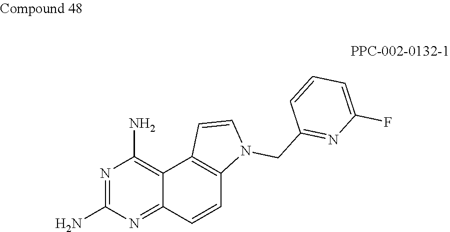 [SMILES: Nc1nc(N)c2c(ccc3c2ccn3Cc2cccc(F)n2)n1]

¹H NMR (400 MHz, DMSO-d₆) δ 8.00-7.86 (m, 2H), 7.77 (d, J=3.0 Hz, 1H), 7.65 (s, 1H), 7.29 (d, J=3.0 Hz, 1H), 7.15 (d, J=8.8 Hz, 1H), 7.09 (dd, J=8.0, 2.4 Hz, 1H), 6.95 (dd, J=8.0, 2.4 Hz, 1H), 6.76 (s, 2H), 5.63 (s, 2H).

MS (ESI): [M+H⁺] 309.33

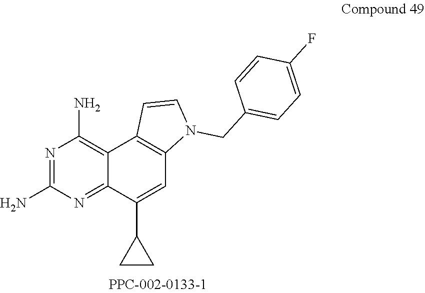 [SMILES: Nc1nc(N)c2c(n1)c(C1CC1)cc1c2ccn1Cc1ccc(F)cc1]

¹H NMR (400 MHz, Methanol-d₄) δ 7.59 (d, J=4.4 Hz, 2H), 7.27-7.15 (m, 2H), 7.13-6.98 (m, 3H), 5.53 (s, 2H), 2.20 (m, 1H), 1.15-1.03 (m, 2H), 0.76-0.65 (m, 2H).

MS (ESI): [M+H⁺] 348.45

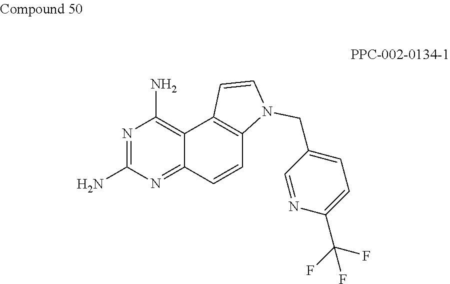 [SMILES: Nc1nc(N)c2c(ccc3c2ccn3Cc2ccc(C(F)(F)F)nc2)n1]

¹H NMR (400 MHz, Chloroform-d+MeOD) δ 8.35 (d, J=2.0 Hz, 1H), 7.45 (m 2H), 7.35 (dd, J=8.4, 2.0 Hz, 1H), 7.28 (s, 1H), 7.03 (d, J=8.4 Hz, 1H), 6.77 (d, J=3.2 Hz, 1H), 5.42 (s, 2H).

MS (ESI): [M+H⁺] 359.30

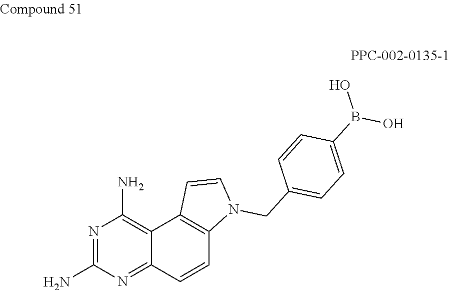 [SMILES: Nc1nc(N)c2c(ccc3c2ccn3Cc2ccc(B(O)O)cc2)n1]

¹H NMR (400 MHz, DMSO-d₆) δ 8.03 (m, 3H), 7.92 (d, J=3.2 Hz, 1H), 7.71 (d, J=7.6 Hz, 2H), 7.45 (s, 2H), 7.38 (d, J=3.2 Hz, 1H), 7.21 (d, J=8.8 Hz, 1H), 7.15 (d, J=7.6 Hz, 2H), 5.59 (s, 2H).

MS (ESI): [M+H⁺] 334.17

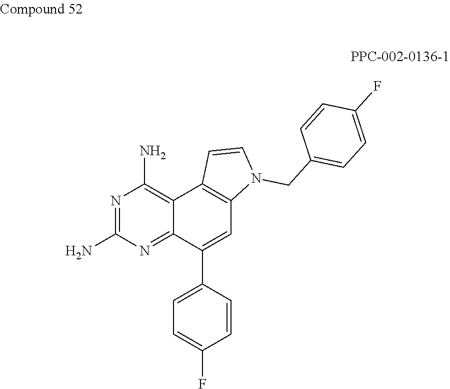 [SMILES: Nc1nc(N)c2c(n1)c(-c1ccc(F)cc1)cc1c2ccn1Cc1ccc(F)cc1]

¹H NMR (400 MHz, Methanol-d₄) δ 7.79 (d, J=0.8 Hz, 1H), 7.69 (d, J=3.2 Hz, 1H), 7.56-7.44 (m, 2H), 7.30-7.19 (m, 4H), 7.16 (dd, J=3.2, 0.8 Hz, 1H), 7.10-7.01 (m, 2H), 5.55 (s, 2H).

MS (ESI): [M+H⁺] 402.40

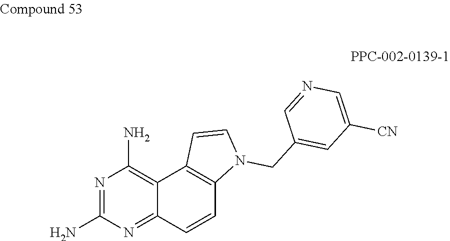 [SMILES: N#Cc1cncc(Cn2ccc3c4c(N)nc(N)nc4ccc32)c1]

¹H NMR (400 MHz, DMSO-d₆) δ 8.94 (d, J=2.0 Hz, 1H), 8.76 (d, J=2.0 Hz, 1H), 8.18 (d, J=2.0 Hz, 1H), 7.94 (d, J=9.0 Hz, 1H), 7.78 (d, J=3.2 Hz, 1H), 7.23 (d, J=3.2 Hz, 1H), 7.11 (d, J=9.0 Hz, 1H), 6.28 (s, 2H), 5.64 (s, 2H).

MS (ESI): [M+H⁺] 316.31

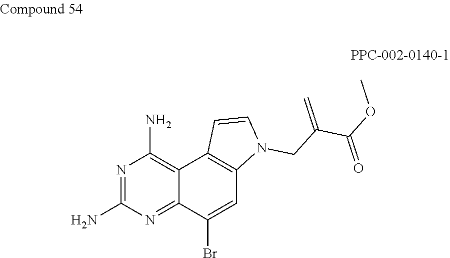 [SMILES: C=C(Cn1ccc2c3c(N)nc(N)nc3c(Br)cc21)C(=O)OC]

¹H NMR (400 MHz, Methanol-d₄) δ 8.11 (d, J=1.2 Hz, 1H), 7.45 (d, J=3.2 Hz, 1H), 6.96 (dd, J=3.2, 0.8 Hz, 1H), 6.27 (d, J=1.0 Hz, 1H), 5.31 (d, J=1.0 Hz, 1H), 5.14 (t, J=1.6 Hz, 2H), 3.79 (s, 3H).

MS (ESI): [M+H⁺] 376.22

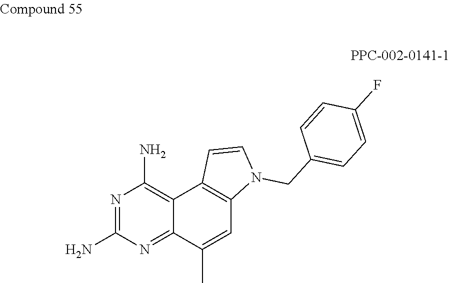 [SMILES: Cc1cc2c(ccn2Cc2ccc(F)cc2)c2c(N)nc(N)nc12]

¹H NMR (400 MHz, Methanol-d₄) δ 7.60 (d, J=3.2 Hz, 1H), 7.41 (t, J=3.2 Hz, 1H), 7.19-7.14 (m, 2H), 7.04 (dd, J=8.8, 3.2 Hz, 2H), 6.95 (t, J=3.2 Hz, 1H), 5.44 (d, J=3.2 Hz, 2H), 2.52 (d, J=3.2 Hz, 3H).

MS (ESI): [M+H⁺] 322.34

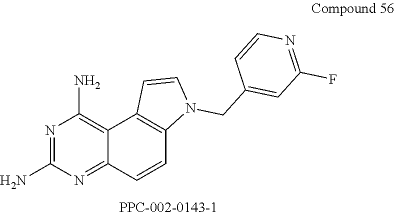 [SMILES: Nc1nc(N)c2c(ccc3c2ccn3Cc2ccnc(F)c2)n1]

¹H NMR (400 MHz, DMSO-d₆) δ 8.16 (d, J=5.2 Hz, 1H), 7.71 (d, J=9.0 Hz, 1H), 7.66 (d, J=3.2 Hz, 1H), 7.18 (d, J=3.2 Hz, 1H), 7.05 (d, J=9.0 Hz, 1H), 7.00-6.98 (m, 1H), 6.84 (d, J=1.5 Hz, 1H), 6.79 (s, 2H), 5.80 (s, 2H), 5.64 (s, 2H).

MS (ESI): [M+H⁺] 309.36

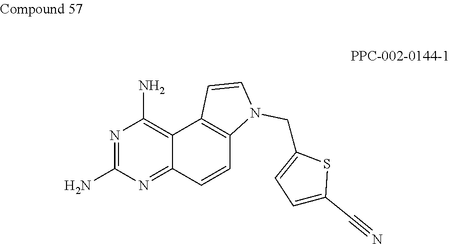 [SMILES: N#Cc1ccc(Cn2ccc3c4c(N)nc(N)nc4ccc32)s1]

¹H NMR (400 MHz, DMSO-d₆) δ 7.91-7.79 (m, 3H), 7.63 (d, J=3.2 Hz, 1H), 7.11 (d, J=3.2 Hz, 1H), 7.05 (d, J=8.8 Hz, 1H), 6.74 (s, 2H), 5.77 (s, 2H), 5.51 (s, 2H).

MS (ESI): [M+H⁺] 321.38

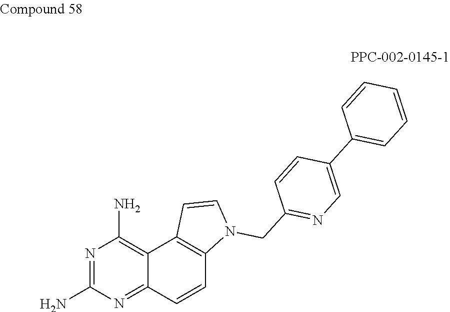 [SMILES: Nc1nc(N)c2c(ccc3c2ccn3Cc2ccc(-c3ccccc3)cn2)n1]

¹H NMR (400 MHz, DMSO-d₆) δ 8.85 (d, J=2.4 Hz, 1H), 8.01 (dd, J=8.4, 2.4 Hz, 1H), 7.78 (d, J=9.2 Hz, 1H), 7.74-7.63 (m, 3H), 7.48 (t, J=7.2 Hz, 2H), 7.41 (t, J=7.2 Hz, 1H), 7.15 (d, J=3.2 Hz, 1H), 7.05 (d, J=8.4 Hz, 2H), 6.82 (s, 2H), 5.83 (s, 2H), 5.65 (s, 2H).

MS (ESI): [M+H⁺] 367.41

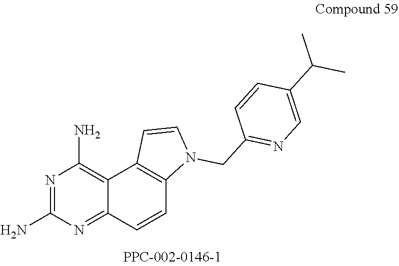 [SMILES: CC(C)c1ccc(Cn2ccc3c4c(N)nc(N)nc4ccc32)nc1]

¹H NMR (400 MHz, DMSO-d₆) δ 8.42 (d, J=2.4 Hz, 1H), 7.77 (d, J=8.8 Hz, 1H), 7.68-7.54 (m, 2H), 7.13 (d, J=3.2 Hz, 1H), 7.04 (d, J=8.8 Hz, 1H), 6.92 (d, J=8.0 Hz, 1H), 6.87 (s, 2H), 5.91 (s, 2H), 5.55 (s, 2H), 2.88 (m, 1H), 1.17 (d, J=6.8 Hz, 6H).

MS (ESI): [M+H⁺] 333.40

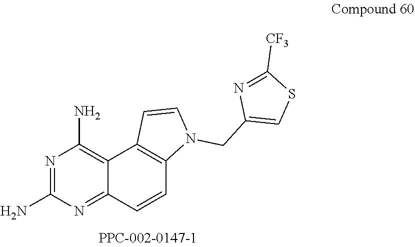 [SMILES: Nc1nc(N)c2c(ccc3c2ccn3Cc2csc(C(F)(F)F)n2)n1]

¹H NMR (400 MHz, DMSO-d₆) δ 7.92-7.82 (m, 2H), 7.62 (d, J=3.2 Hz, 1H), 7.13 (d, J=3.2 Hz, 1H), 7.07 (d, J=9.2 Hz, 1H), 6.83 (s, 2H), 5.87 (s, 2H), 5.71 (s, 2H).

MS (ESI): [M+H⁺] 365.33

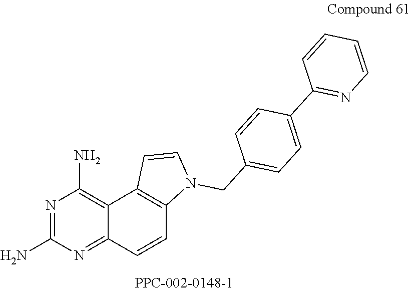 [SMILES: Nc1nc(N)c2c(ccc3c2ccn3Cc2ccc(-c3ccccn3)cc2)n1]

¹H NMR (400 MHz, DMSO-d₆) δ 8.63 (dt, J=4.8, 1.6 Hz, 1H), 8.06-7.98 (m, 2H), 7.94-7.81 (m, 2H), 7.78 (d, J=9.0 Hz, 1H), 7.69 (d, J=3.2 Hz, 1H), 7.35-7.25 (m, 3H), 7.15 (d, J=3.2 Hz, 1H), 7.05 (d, J=9.0 Hz, 1H), 6.85 (s, 2H), 5.93-5.80 (m, 2H), 5.58 (s, 2H).

MS (ESI): [M+H⁺] 367.41

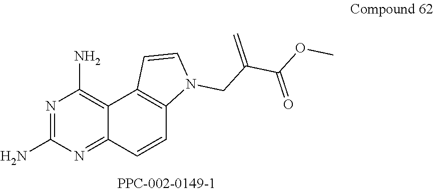 [SMILES: C=C(Cn1ccc2c3c(N)nc(N)nc3ccc21)C(=O)OC]

¹H NMR (400 MHz, DMSO-d₆) δ 7.71 (d, J=8.8 Hz, 1H), 7.45 (d, J=3.2 Hz, 1H), 7.14-6.99 (m, 2H), 6.66 (s, 2H), 6.15 (s, 1H), 5.67 (s, 2H), 5.24 (s, 1H), 5.14 (s, 2H), 3.73 (s, 3H).

MS (ESI): [M+H⁺] 298.11

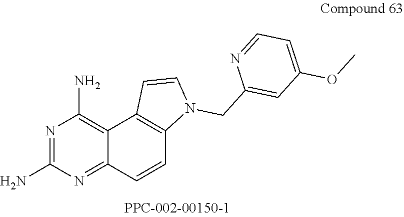 [SMILES: COc1ccnc(Cn2ccc3c4c(N)nc(N)nc4ccc32)c1]

¹H NMR (400 MHz, DMSO-d₆) δ 8.47-8.25 (m, 1H), 7.91-7.55 (m, 2H), 7.01 (m, 5H), 6.59 (s, 1H), 6.04 (s, 2H), 5.52 (s, 2H), 3.73 (s, 3H).

MS (ESI): [M+H⁺] 321.20.

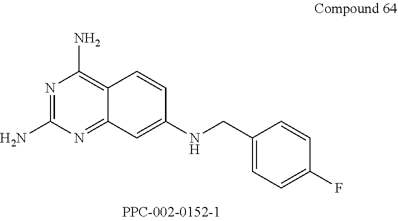 [SMILES: Nc1nc(N)c2ccc(NCc3ccc(F)cc3)cc2n1]

¹H NMR (400 MHz, DMSO-d₆) δ 7.83 (s, 2H), 7.51-7.38 (m, 2H), 7.19-7.11 (m, 4H), 7.08 (s, 1H), 6.55 (s, 2H), 6.29 (t, J=6.4 Hz, 1H), 4.29 (d, J=6.0 Hz, 2H).

MS (ESI): [M+H⁺] 284.30

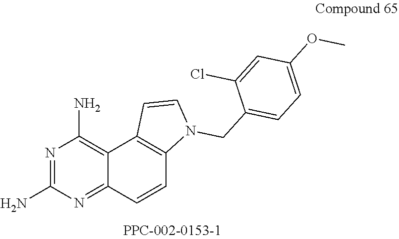 [SMILES: COc1ccc(Cn2ccc3c4c(N)nc(N)nc4ccc32)c(Cl)c1]

¹H NMR (400 MHz, DMSO-d₆) δ 7.70 (d, J=9.2 Hz, 1H), 7.51 (d, J=3.2 Hz, 1H), 7.11 (dd, J=8.0, 2.8 Hz, 2H), 7.05 (d, J=9.2 Hz, 1H), 6.84 (dd, J=8.4, 2.4 Hz, 1H), 6.76 (s, 2H), 6.73 (d, J=8.4 Hz, 1H), 5.78 (s, 2H), 5.50 (s, 2H), 3.74 (s, 3H).

MS (ESI): [M+H⁺] 354.79

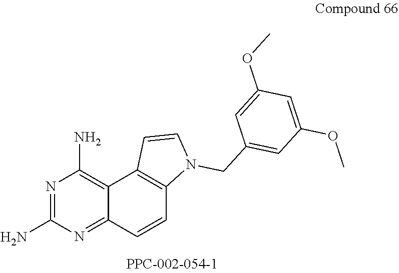 [SMILES: COc1cc(Cn2ccc3c4c(N)nc(N)nc4ccc32)cc(OC)c1]

¹H NMR (400 MHz, DMSO-d₆) δ 7.74 (dd, J=9.0, 0.8 Hz, 1H), 7.62 (d, J=3.2 Hz, 1H), 7.13-7.08 (m, 1H), 7.04 (d, J=9.0 Hz, 1H), 6.75 (s, 2H), 6.38 (t, J=2.4 Hz, 1H), 6.34 (d, J=2.4 Hz, 2H), 5.76 (s, 2H), 5.41 (s, 2H), 3.67 (s, 6H).

MS (ESI): [M+H⁺] 350.34

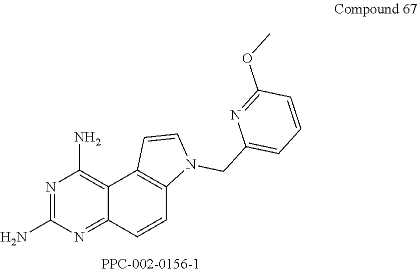 [SMILES: COc1cccc(Cn2ccc3c4c(N)nc(N)nc4ccc32)n1]

¹H NMR (400 MHz, DMSO-d₆) δ 7.79 (dd, J=9.0, 0.8 Hz, 1H), 7.69-7.50 (m, 2H), 7.18-7.09 (m, 1H), 7.05 (d, J=8.8 Hz, 1H), 6.78 (s, 2H), 6.69 (dd, J=8.8, 0.8 Hz, 1H), 6.51 (dd, J=7.2, 0.8 Hz, 1H), 5.80 (s, 2H), 5.49 (s, 2H), 3.82 (s, 3H).

MS (ESI): [M+H⁺] 321.37

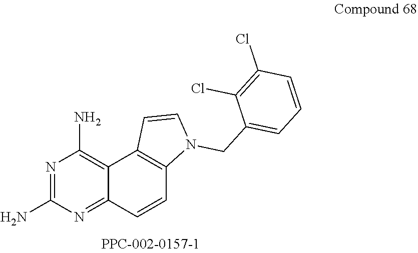 [SMILES: Nc1nc(N)c2c(ccc3c2ccn3Cc2cccc(Cl)c2Cl)n1]

¹H NMR (400 MHz, DMSO-d₆) δ 7.67 (d, J=9.0 Hz, 1H), 7.62-7.51 (m, 2H), 7.33-7.13 (m, 2H), 7.04 (d, J=9.0 Hz, 1H), 6.76 (s, 2H), 6.39 (dd, J=7.6, 1.2 Hz, 1H), 5.76 (s, 2H), 5.65 (s, 2H).

MS (ESI): [M+H⁺] 359.21

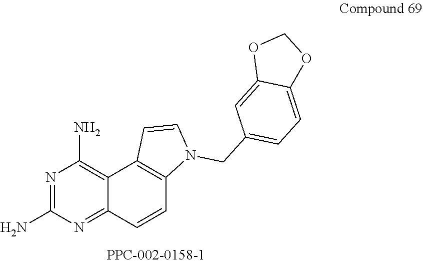 [SMILES: Nc1nc(N)c2c(ccc3c2ccn3Cc2ccc3c(c2)OCO3)n1]

¹H NMR (400 MHz, DMSO-d₆) δ 7.78 (d, J=8.8 Hz, 1H), 7.62 (d, J=3.0 Hz, 1H), 7.08 (d, J=3.0 Hz, 1H), 7.03 (d, J=8.8 Hz, 1H), 6.88-6.79 (m, 2H), 6.76-6.73 (m, 1H), 5.96 (s, 2H), 5.76 (s, 2H), 5.38 (s, 2H).

MS (ESI): [M+H⁺] 334.36

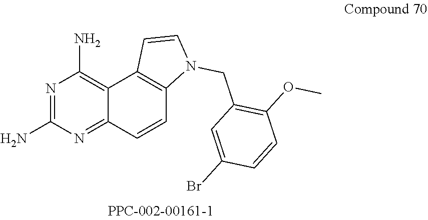 [SMILES: COc1ccc(Br)cc1Cn1ccc2c3c(N)nc(N)nc3ccc21]

¹H NMR (400 MHz, DMSO-d₆) δ 7.71 (d, J=8.8 Hz, 1H), 7.55 (d, J=3.2 Hz, 1H), 7.43 (dd, J=8.8, 2.4 Hz, 1H), 7.10 (d, J=3.2 Hz, 1H), 7.07-7.01 (m, 2H), 6.79 (d, J=2.4 Hz, 1H), 6.73 (s, 2H), 5.75 (s, 2H), 5.42 (s, 2H), 3.87 (s, 3H).

MS (ESI): [M+H⁺] 398.24.

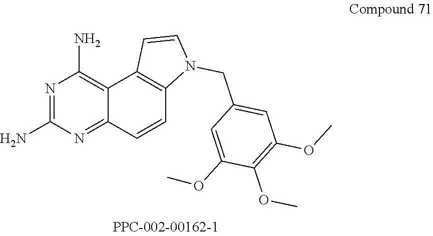 [SMILES: COc1cc(Cn2ccc3c4c(N)nc(N)nc4ccc32)cc(OC)c1OC]

¹H NMR (400 MHz, DMSO-d₆) δ 7.83 (d, J=9.2 Hz, 1H), 7.64 (d, J=3.2 Hz, 1H), 7.09 (d, J=3.2 Hz, 1H), 7.04 (d, J=9.2 Hz, 1H), 6.74 (s, 2H), 6.61 (s, 2H), 5.76 (s, 2H), 5.38 (s, 2H), 3.68 (s, 6H), 3.59 (s, 3H).

MS (ESI): [M+H+] 398.24

### Anticancer Properties of Compound 1 (IRS-17)

The anticancer properties of Compound 1 were investigated as follows:

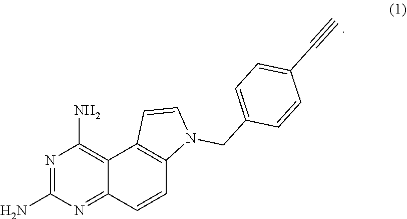 [SMILES: C#Cc1ccc(Cn2ccc3c4c(N)nc(N)nc4ccc32)cc1]

### A. Compound 1—Purine and Thymidine Inhibition

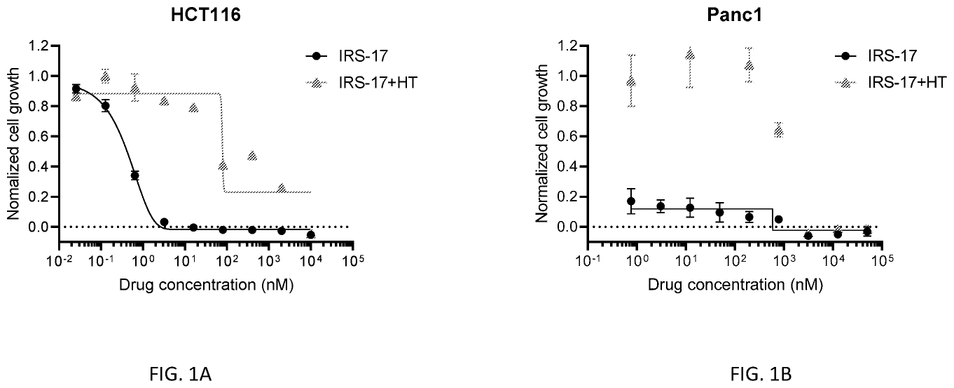

Cells of (a) HCT116 and (b) Panc1 cell lines were each seeded in 96-well plates at the density of 3000/well in 80 μL of DMEM media supplemented with 10 vol % dialyzed FBS. The next day, 10 μL of media with or without hypoxanthine (1 mM) and thymidine (160 μM) was added to each well. 10 μL of Compound 1 diluted in media as 10× stock of desired concentration was also added. Relative cell number is measured by resazurin right before and 4 days after the addition of Compound 1. Namely, resazurin dissolved in phosphate-buffered saline (PBS) was added to each well to a final concentration of 10 μg/mL followed by 1-hour incubation at 37° C. Fluorescence (excitation 550 nm, emission 590 nm) is then measured by a microplate reader. The results are provided in FIGS. 1A and 1B. As illustrated in FIGS. 1A and 1, Compound 1 inhibited cell growth under supplement and non-supplement conditions. Inhibition of growth in both cancer cell lines was achieved via disruption of purine and thymine synthesis.

### B. Compound 1—Folate Competition

Compound 1 was tested relative to pemetrexed and methotrexate against two isogenic CHO cell lines with low and high RFC expression levels. IC50 values were determined in folic acid free RPMI media supplemented with 10 vol. % dialyzed FBS and 25 nM 5-formyl THF. Table II provides the IC50 values for Compound 1, Pemetrexed and Methotrexate.

TABLE II

IC50 (nM)

| | Low RFC expression | High RFC expression |
|---|---|---|
| Compound 1 | <0.002 | 0.06 |
| Pemetrexed | 800 | 150 |
| Methotrexate | 4000 | 480 |

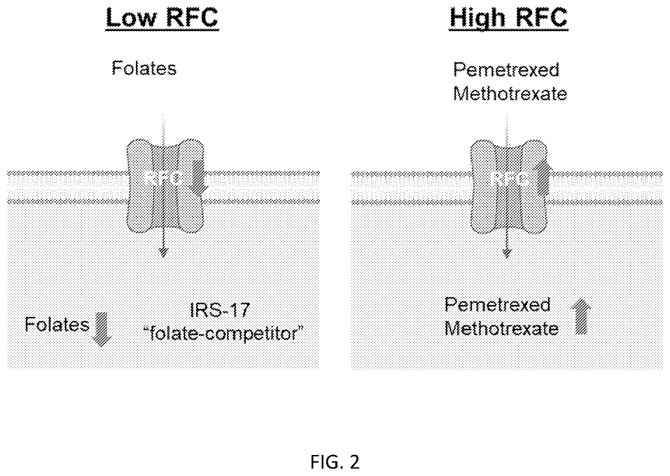

As provided in Table II, Compound 1 exhibits higher potency in cells down-regulating RFC, which are resistant to Pemetrexed and Methotrexate. FIG. 2 also illustrates the higher potency of Compound 1 in low-RFC expression cells.

### C. Effect of Compound 1 on KPC Pancreatic Ductal Adenocarcinoma Allograft

Tumor harvested from K-ras^{LSL.G12D/+}; Trp53^{R172H/+}; Pdx-1-Cre (KPC) mice was passaged at the flank of C57BL/6 subcutaneously. To initiate allografts, subcutaneous tumors were harvested and minced in DMEM media, mixed with equal volume of Matrigel basement membrane matrix (Corning 354234), and injected subcutaneously at the flank of male C57BL/6 mice. Tumors were measured twice per week. Once the average tumor volume reaches 100 mm³, Compound 1 (5 mg/kg body weight, dissolved in 10% 2-hydroxypropyl-β-cyclodextrin) and vehicle (10% 2-hydroxypropyl-β-cyclodextrin, 10 mL/kg body weight) were injected intraperitoneally once daily, with continued tumor monitoring. At the end of experiment, mice were euthanized by cervical dislocation after being fasted for 6 hr. Tumors are collected into aluminum foil, clamped by a Wollenberg clamp pre-cooled in liquid N₂, and kept cold in liquid N₂.

To extract metabolites from tumor samples, tumors were first ground by a Cryomill (Retsch). About 10 mg of ground tissue is measured and extracted by extraction buffer (acetonitrile:methanol:H₂O=2:2:1, then supplemented with 0.5 vol % formic acid) at the ratio of 370 μL buffer per 10 mg tissue. Samples were vortexed and adjusted to pH7 by 15% NH₄HCO₃ (30 μL per 10 mg tissue). Samples were centrifuged (16000 g for 20 min) at 4° C., and supernatants are collected for measurement. Metabolite levels were measured by Q Exactive Plus Hybrid Quadrupole-Orbitrap coupled with hydrophobic interaction chromatography (HILIC) operating in the negative ionization mode.

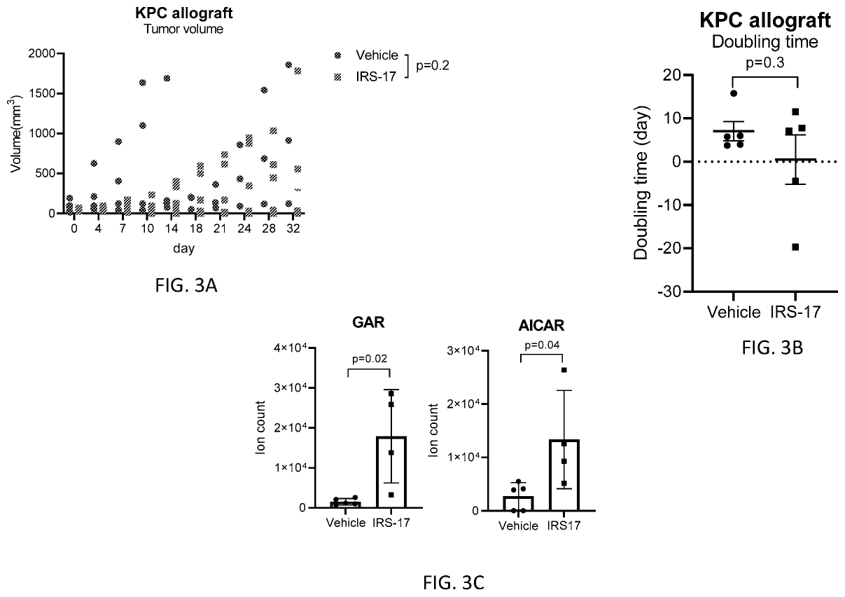

FIG. 3A illustrates change in tumor volume over time, and FIG. 3B illustrates tumor doubling time. As provided in FIGS. 3A and 3B, Compound 1 substantially inhibited tumor growth over the study time period, and protracted tumor doubling time. FIG. 3C illustrates levels of intermediates in purine synthesis glycineamide ribonucleotide (GAR) and 5-aminoimidazole-4-carboxamide ribonucleotide (AlCAR) in the tumors, indicating folate competition or interference.

### D. Effect of Compound 1 on HCT116 Colorectal Carcinoma Xenograft

To initiate allografts, 2×10⁶ MC38 cells were injected subcutaneously at the flank of female C57BL/6 mice. Once the average tumor volume reaches 50 mm³, Compound 1 (5 mg/kg body weight, dissolved in 10% 2-hydroxypropyl-β-cyclodextrin) and vehicle (10% 2-hydroxypropyl-β-cyclodextrin, 10 mL/kg body weight) are injected intraperitoneally once daily. After the final dose, mice were fasted for 12 hr. Then blood was collected by tail snip into a tube without anticoagulants. Serum was collected by taking the supernatant after centrifugation. Mice were then euthanized by cervical dislocation, and tumors were harvested and frozen in liquid N₂. Tumor metabolites were extracted and measured as previously described in C. To extract serum metabolites, 2.5 μL of serum was added into 80 μL of methanol. Samples were centrifuged (16000 g for 10 min) at 4° C., and supernatant is collected.

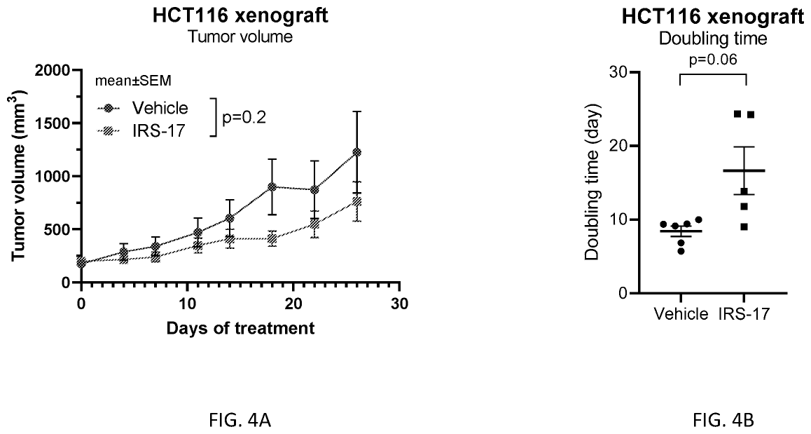

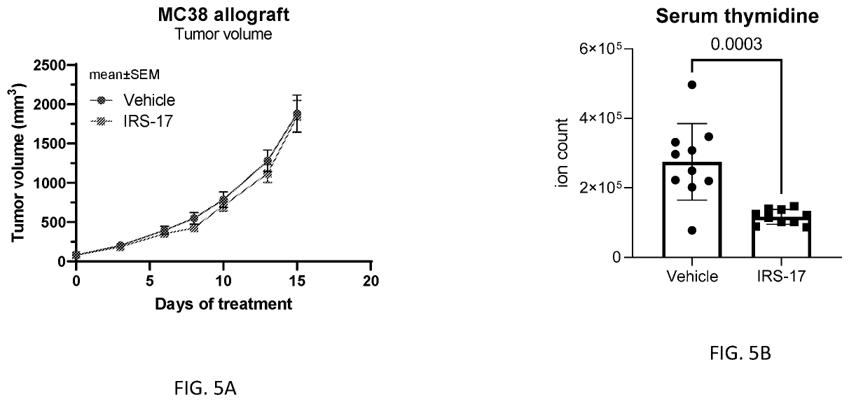

FIG. 4A illustrates change in tumor volume over time, and FIG. 4B illustrates tumor doubling time. As provided in FIGS. 4A and 4B, Compound 1 substantially inhibited tumor growth over the study time period, and protracted tumor doubling time.

### E. Effect of Compound 1 on MC38 Mouse Colon Adenocarcinoma Allograft

To initiate allografts, 2×10⁶ MC38 cells were injected subcutaneously at the flank of female C57BL/6 mice. Once the average tumor volume reaches 50 mm³, IRS-17 (5 mg/kg body weight, dissolved in 10% 2-hydroxypropyl-β-cyclodextrin) and vehicle (10% 2-hydroxypropyl-β-cyclodextrin, 10 mL/kg body weight) are injected intraperitoneally once daily. After the final dose, mice were fasted for 12 hr. Then blood was collected by tail snip into a tube without anticoagulants. Serum was collected by taking the supernatant after centrifugation. Mice were then euthanized by cervical dislocation, and tumors are harvested and frozen in liquid N₂. Tumor metabolites were extracted and measured as previously described in C. To extract serum metabolites, 2.5 μL of serum was added into 80 μL of methanol. Samples were centrifuged (16000 g for 10 min) at 4° C., and supernatant is collected.

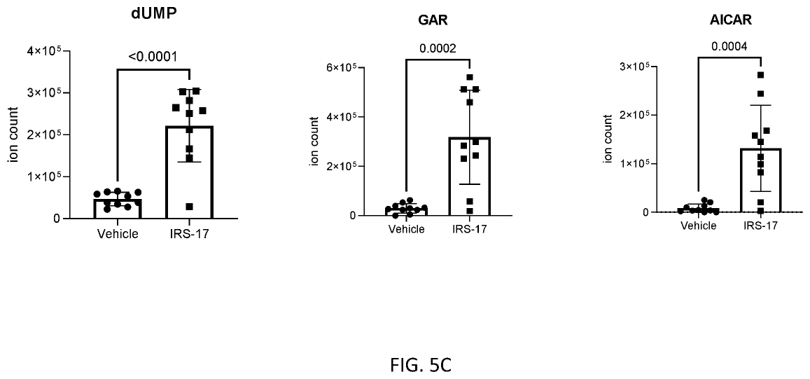

FIG. 5A illustrates some reduction in tumor volume over the treatment period. FIG. 5B illustrates pool size of circulating thymidine, and FIG. 5C illustrates thymidine ribonucleotide intermediate dUMP and purine intermediates GAR and AlCAR levels in the tumors in response to administration of Compound 1.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A pharmaceutical composition comprising a compound of Formula (I) and/or salts thereof: 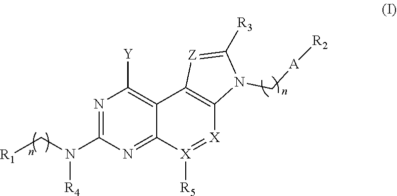
wherein R₁, R₃, R₄ and R₅ are independently selected from the group consisting of hydrogen, alkyl, alkenyl, heteroalkyl, cycloalkyl, heterocycloalkyl, aryl, heteroaryl, imine, cyanoimine, alkylene-aryl, alkylene-heteroaryl, amide, sulfonamide, acid, halo, and urea, wherein the alkyl, alkenyl, heteroalkyl, cycloalkyl, heterocycloalkyl, aryl, heteroaryl, alkylene-aryl, alkylene-heteroaryl, amide and sulfonamide are optionally substituted with one or more substituents selected from the group consisting of (C₁-C₁₀)-alkyl, (C₁-C₁₀)-alkenyl, cycloalkyl, heterocycloalkyl, aryl, heteroaryl, alkoxy, amide, sulfonamide, urea, halo, cyano, hydroxy, C(O)OR₆, and C(O)R₇, wherein R₆ is selected from the group consisting of hydrogen, alkyl and alkenyl and R₇ is selected from the group consisting of hydrogen, alkyl, alkenyl, heteroalkyl, cycloalkyl, heterocycloalkyl, aryl, heteroaryl and NR₈R₉, wherein R₈ and R₉ are independently selected from the group consisting of hydrogen, alkyl, alkenyl, aryl and heteroaryl; and
wherein R₂ is selected from the group consisting of alkyl, cycloalkyl, heterocycloalkyl, alkynyl, alkenyl, alkylnylene-alkyl, alkynylene-cycloalkyl, alkynylene-heterocycloalkyl, alkynylene-aryl, alkynylene-heteroaryl, alkynylene-amine, alkynylene-protected amine, alkynylene-alkylsilane, fluoroalkyl, fluoro, bromo, B(OH)₂, nitro, cyano, and alkoxy; and
wherein A is selected from the group consisting of aryl and heteroaryl; and
wherein X and Z are independently selected from the group consisting of C, N, O, S, SO₂, and NR₁₀R₁₁, wherein R₁₀ and R₁₁ are independently selected from the group consisting of hydrogen, alkyl, heteroalkyl, cycloalkyl, heterocycloalkyl, alkenyl, aryl, heteroaryl, amide, sulfonamide, urea and C(O)R₁₂ wherein R₁₂ is selected from the group consisting of hydrogen, alkyl, alkenyl, heteroalkyl, cycloalkyl, heterocycloalkyl, aryl and heteroaryl and wherein R₁₀ and R₁₁ may optionally form a ring structure; and
wherein Y is selected from the group consisting of OH, alkoxy, and NR₁₃R₁₄, wherein R₁₃ and R₁₄ are independently selected from the group consisting of hydrogen, alkyl, heteroalkyl, cycloalkyl, heterocycloalkyl, alkenyl, aryl, heteroaryl, amide, sulfonamide, urea, alkylene-aryl, alkylene-heteroaryl, and C(O)Ris wherein R₁₅ is selected from the group consisting of hydrogen, alkyl, alkenyl, heteroalkyl, cycloalkyl, heterocycloalkyl, aryl and heteroaryl and wherein R₁₃ and R₁₄ may optionally form a ring structure, wherein the aryl, heteroaryl, alkylene-aryl and alkylene heteroaryl are optionally substituted with one or more substituents selected from the group consisting of alkyl, alkenyl, alkynyl, halo, and alkynylene-alkylsilane; and
n is an integer from 0 to 5,
wherein the compound of Formula (I) is present in the pharmaceutical composition in an amount sufficient to exhibit anticancer properties.

2. The pharmaceutical composition of claim 1, wherein R₂ is selected from the group consisting of alkynyl, alkylnylene-alkyl, alkynylene-cycloalkyl, alkynylene-heterocycloalkyl, alkynylene-aryl, alkynylene-heteroaryl, alkynylene-amine, alkynylene-protected amine, and alkynylene-alkylsilane.

3. The pharmaceutical composition of claim 1, wherein R₂ is selected from the group consisting of alkynyl, alkenyl, and alkylnylene-alkyl.

4. The pharmaceutical composition of claim 2, wherein R₁ and R₄ are independently selected from the group consisting of hydrogen, alkyl, cycloalkyl, and heterocycloalkyl.

5. The pharmaceutical composition of claim 2, wherein Y is NR₁₂R₁₃, wherein R₁₂ and R₁₃ are independently selected from the group consisting of hydrogen, alkyl, heteroalkyl, cycloalkyl, heterocycloalkyl, alkenyl, aryl, and heteroaryl.

6. The pharmaceutical composition of claim 2, wherein X and Z are independently selected from C and N.

7. The pharmaceutical composition of claim 2, wherein R₃ is selected from the group consisting of hydrogen and alkyl.

8. The pharmaceutical composition of claim 2, wherein the compound of Formula (I) is:

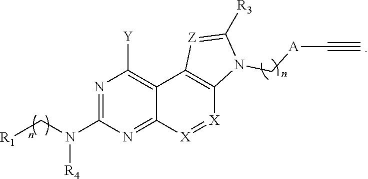

9. The pharmaceutical composition of claim 8, wherein X and Z are selected from the group consisting of C and N, and wherein R₁, R₃, and R₄ are independently selected from the group consisting of hydrogen, alkyl, cycloalkyl, and heterocycloalkyl.

10. The pharmaceutical composition of claim 2, wherein the compound of Formula (I) and/or salt thereof is present in an amount of 0.01 nM to 1 μM.

11. The pharmaceutical composition of claim 2, wherein the compound of Formula (I) and/or salt thereof is present in an amount of 0.1 nM to 500 nM.

12. The pharmaceutical composition of claim 2, wherein the compound of Formula (I) inhibits purine and/or thymine biosynthesis in cancer cells.

13. The pharmaceutical composition of claim 12, wherein the cancer cells are pancreatic cancer cells.

14. The pharmaceutical composition of claim 2, wherein the compound of Formula (I) exhibits higher potency relative to pemetrexed or methotrexate for cancer cells down-regulating reduced folate carrier.

15. The pharmaceutical composition of claim 2, wherein the compound of Formula (I) inhibits colorectal tumor growth.

16. The pharmaceutical composition of claim 2 further comprising one more adjuvants or additional chemotherapeutic agents.

17. A method of treating cancer comprising:
administering to a patient having cancerous tissue the pharmaceutical composition of claim 1.

18. The method of claim 17, wherein cells of the cancerous tissue down-regulate reduced folate carrier.

19. The method of claim 18, wherein IC50 of the compound of Formula (I) is less than 0.1 nM.

20. The method of claim 18, wherein the compound of Formula (I) is: [chemical structure]
